(19) United States
(12) Patent Application Publication
(10) Pub. No.: US 2026/0346445 A1
(43) Pub. Date: Nov. 5, 2026

(54) Hybrid Compounded Robotic Platform

(71) Applicant: Edel Demeke Beyene, Sheridan, WY (US)

(72) Inventor: Edel Demeke Beyene, Sheridan, WY (US)

(21) Appl. No.: 19/360,024

(22) Filed: Oct. 16, 2025

Related U.S. Application Data
(63) Continuation-in-part of application No. 19/063,386, filed on Feb. 26, 2025, now abandoned.

(51) Int. Cl. B25J 9/16; A61B 34/30

(52) U.S. Cl. CPC B25J 9/1664; A61B 34/30; A61B 2034/305

(57) ABSTRACT

A hybrid compounded robotic platform is disclosed, comprising a mobile or immobile support base carrying a robotic manipulator arm with a wrist assembly. A primary end-effector is detachably coupled to the wrist via a multi-signal coupler providing mechanical, electrical, and fluidic connections. The primary end-effector supports a secondary end-effector, and the secondary supports a tertiary end-effector, each with independent actuation and coupled via respective multi-signal couplers. An integrated parking station with shaped docking seats, alignment fixtures, and electrical contacts stores, charges, and calibrates end-effectors when not in use. An intelligent management unit sequences tool exchanges, monitors stability and collision-avoidance sensors, and controls docking operations. The platform may operate individually or in coordinated multi-platform systems under a central server, enabling automated task execution, resource sharing, and dynamic reconfiguration. This modular design provides adaptability, rapid tool change, and multi-role capability across industrial, medical, logistics, and field environments.

### CROSS-REFERENCE TO RELATED APPLICATIONS

This application claims benefit to the earlier filing date and right of priority under 35 U.S.C. § 120 to US provisional application 63/663,917 filed on Jun. 25, 2024, US non-provisional application Ser. No. 19/063,386 filed Feb. 26, 2025, and PCT application number PCT/IB2025/05645 filed on Jun. 25, 2025, the contents of which are herein incorporated by reference under 37 CFR 1.57(b).

### BACKGROUND OF THE INVENTION

### Technical Field

This invention relates generally to a versatile hybrid compounded robotic platform. This invention relates more particularly to an apparatus/device for providing modular, reconfigurable, and adaptive robotic systems capable of performing multiple functions across diverse environments and industries.

This invention relates generally to apparatuses and devices for multi-modal robotic platforms that incorporate interchangeable modules, multiple degrees of freedom, and adaptable locomotion mechanisms to support varied operational tasks. This method also can be used with autonomous, semi-autonomous, or manually controlled robotic systems for healthcare, manufacturing, hospitality, logistics, construction, mining, agriculture, exploration, and military applications.

This invention relates more particularly to a reconfigurable robotic platform to seamlessly transition between different support bases, end-effectors, and control modes to perform distinct operational roles without substantial redesign or modification.

Field of the Invention: The field of art to which this invention pertains includes robotics and automation, specifically modular robotic platforms configured for adaptability in multiple operational contexts. The subject matter encompasses systems and methods involving mechanical, electrical, and control subsystems for robotic apparatuses with interchangeable components, multi-axis mobility, and configurable task-specific tools. These systems are classified within the technical scope of robots having modular construction and reconfigurable architectures, incorporating elements for autonomous or semi-autonomous operation, coordinated multi-robot systems, and integrated sensing and control for task execution in varied industrial, commercial, and field environments.

### Background Art

Robotic systems have advanced significantly in recent years, finding applications in industries such as healthcare, manufacturing, hospitality, logistics, construction, mining, agriculture, exploration, and military operations. Existing systems are often designed for specialized tasks, making them efficient in narrow applications but limited in versatility. When new functions are required, these robots frequently necessitate substantial mechanical redesigns, software modifications, or the acquisition of entirely separate platforms. This lack of adaptability results in increased operational costs, longer deployment timelines, and inefficiencies in environments that require rapid response to changing demands.

Many industrial robotic systems are purpose-built for fixed installations, such as assembly lines, precision machining, or automated inspection. While effective in their intended roles, such robots lack the ability to adapt to different operational settings or tasks without significant modification. Similarly, healthcare robots may be optimized for patient mobility assistance, surgery, diagnostics, or rehabilitation, but rarely for more than one of these functions. This creates a fragmentation of capabilities, requiring multiple systems to handle different stages of a workflow.

In the hospitality sector, robots are often designed for single functions such as delivering food, cleaning rooms, or interacting with guests. These robots are typically confined to specific environments and lack the mobility or modularity to function effectively across varied operational zones. In logistics, existing solutions focus on discrete tasks—like sorting packages, transporting pallets, or inventory scanning—but rarely combine multiple capabilities into a single adaptable platform.

In construction and field operations, robotic solutions often address specialized needs such as bricklaying, excavation, or heavy-load assistance. However, these systems tend to be inflexible, limiting their applicability across different terrain types, scales of operation, or project requirements. Military and exploration robots face similar challenges, with most platforms being mission-specific and ill-suited for rapid adaptation to new roles in dynamic environments.

The common limitations in these prior art systems include the lack of a unified platform capable of accommodating multiple mobility modes, interchangeable toolsets, and adaptable control systems. Without such versatility, organizations must invest in multiple robotic units for varied functions, increasing space requirements, maintenance complexity, and costs. Furthermore, existing systems often lack integrated coordination capabilities that would allow multiple robots to work together seamlessly, further limiting their effectiveness in complex or large-scale operations. In light of the foregoing prior art, there is a need for a versatile, modular, and reconfigurable robotic platform to better perform a wide array of tasks across diverse environments without requiring substantial redesign or the use of multiple dedicated systems.

### BRIEF SUMMARY OF THE INVENTION

According to a first aspect of the invention, there is a Hybrid Compounded Robotic Platform comprising a support architecture that may be mobile, immobile, or a detachable combination thereof; at least one robotic manipulator arm mounted to the support architecture and terminating in a wrist assembly; and a chain of modular end-effectors including primary, secondary, and tertiary modules, each detachably coupled via a multi-signal coupler providing mechanical, electrical, and fluidic connections. The primary end-effector includes actuators configured to drive the secondary module, and the secondary includes actuators configured to drive the tertiary module. The platform further comprises an intelligent management unit in communication with the manipulator, couplers, and docking systems, programmed to sequence attachment and detachment operations, monitor power and balance, compensate for motion changes, and command automatic calibration, self-testing, and power-module swapping.

Further features of this aspect include multi-signal couplers incorporating mechanical latches, electrical contacts, and fluidic conduits that automatically mate on engagement, with alignment pins and tapered guides ensuring precise orientation. Each end-effector can include one or more degrees of freedom driven by dedicated motors, providing independent actuation beyond the primary manipulator arm. The tertiary end-effectors may include medical imaging or treatment devices, manufacturing or logistics tools, or collaborative robotic arms for specialized applications.

According to a second aspect of the present invention, there is a Hybrid Compounded Robotic Platform in the form of a docking and tool management system, wherein the support architecture includes one or more parking stations either integrated with the base or implemented externally and networked. Each station includes shaped docking bays matching the geometry of the primary, secondary, and tertiary end-effectors, with alignment fixtures, passive pins, and spring-loaded contacts for electrical and fluidic connectivity. The docking system may further include an autonomous power-module swapping mechanism triggered by the management unit. An advantage of this object is that it provides continuous operational readiness through automated calibration and charging cycles without manual intervention.

According to a third aspect of the invention, there is a Hybrid Compounded Robotic Platform configured with an intelligent management unit implementing algorithms to dynamically compensate for center-of-mass shifts as modules are exchanged. The control system adjusts base drive and joint torques in real time, maintaining balance and stability during reconfiguration. An advantage of this aspect is that it enhances precision and safety, enabling tool exchange during live operations without external stabilization.

According to a fourth aspect of the invention, there is a Hybrid Compounded Robotic Platform including a detachable coupling interface that allows an immobile base to be mounted on a mobile base to form a combined operating platform and later detached for redeployment. The manipulator arm may be configured as a forearm-only assembly comprising an elbow and wrist directly connected to the base, or may include a collaborative robot arm mounted to a lifting column. An advantage of this arrangement is compactness and portability, allowing reconfiguration between stationary precision tasks and mobile field operations.

According to a fifth aspect of the invention, there is a Hybrid Compounded Robotic Platform further comprising a base-to-base multi-signal connector enabling multiple platforms to be physically coupled at their bases for sharing of power, data, and fluidic lines. The connector incorporates inter-platform alignment latches and position guides to ensure correct engagement. This arrangement supports cooperative multi-robot tasks where platforms operate as a unified system, for example, carrying large objects or supporting a shared workpiece.

According to a sixth aspect of the invention, there is a System of Robotic Platforms comprising a plurality of hybrid compounded robotic platforms under the supervision of a central coordination server. The server communicates with each intelligent management unit to allocate tasks, synchronize tool exchanges, and resolve potential collision risks by maintaining a shared dynamic map of all active platforms, bases, and docking stations.

Further features of this system include server-based management of tool inventories and parking station statuses, autonomous delivery of designated modules from one platform to another, and control of cooperative operations including temporary base-to-base coupling. An advantage of this aspect is scalability: multiple platforms may perform interdependent operations with coordinated timing and shared resources, increasing throughput and operational efficiency.

According to a seventh aspect of the invention, there is a Method of Automated Task Execution employing a hybrid compounded robotic platform. The method comprises receiving a structured task specification identifying the required end-effector or workpiece and its target location; commanding the management unit to detach non-essential modules and dock them; retrieving and coupling the required end-effectors in order; compensating for mass changes; performing the task; and returning the modules for calibration and charging upon completion.

Further features of this method include voice-command initiation and natural-language parsing, inter-platform exchanges directed by the coordination server, and synchronized power-module replacement routines. The architecture may also redeploy immobile modules onto mobile bases for new task assignments. An advantage of this method is that it unifies tool management, movement control, and task planning into an automated cycle, reducing downtime and enabling hands-free robotic operations across diverse domains.

According to an eighth aspect of the invention, there is a Hybrid Compounded Robotic Platform wherein the support base is omni-form, accommodating and stowing end-effectors or workpieces of differing geometries, such as ring-type imaging devices or patient-support structures. This configuration enables modular docking, transport, and reconfiguration as part of automated workflows.

The present invention provides a comprehensive modular robotic system that integrates compound tool chains, autonomous docking, and networked coordination.

The architecture's hybrid mobility—combining stationary precision with mobile reach—addresses prior art limitations in flexibility and reusability.

Intelligent real-time control ensures stability, allowing reconfiguration under load and enabling safe use in sensitive environments such as medical, aerospace, or industrial settings.

Automated docking and calibration reduce human intervention and operational latency, while the shared coordination server supports multi-platform cooperation and fleet-level orchestration.

Overall, the inventive concept provides a scalable, adaptable, and self-managing robotic ecosystem that achieves continuous, reliable operation with reduced equipment redundancy and increased efficiency across multiple fields of use.

The invention will now be described, by way of example only, with reference to the accompanying drawings.

### BRIEF DESCRIPTION OF THE SEVERAL VIEWS OF THE DRAWINGS

The accompanying drawings, which are incorporated in and form a part of this specification, illustrate exemplary embodiments of the invention and, together with the description, serve to explain the principles of the invention. The drawings are not necessarily to scale, and certain features may be exaggerated or simplified for clarity of explanation.

FIG. 1 is a perspective view of an exemplary hybrid compounded robotic platform (HCRP) with a fixed support base. This view illustrates the robotic platform's general configuration, including a support column base mounting a multi-jointed manipulator arm and an attached modular end-effector system. The platform's quick-connect interfaces for tools and extensions are visible, as well as a docking station on the base for storing and servicing additional end-effectors.

FIG. 2 is a perspective view of the robotic platform with a mobile support base configuration. In this embodiment, the platform is built on a wheeled (or otherwise movable) base, demonstrating an alternate form of the support structure. The modular manipulator arm and stackable end-effector system are similarly shown, highlighting that the platform's interchangeable tool architecture is applicable to a mobile unit for maneuverability.

FIGS. 3A and 3B illustrate details of the manipulator arm and tool coupling mechanism. FIG. 3A shows a conventional multi-axis industrial robotic arm (prior art) with its typical articulated joints and defined axes, providing context for the improvements of the present platform. FIG. 3B is an enlarged detail view of the HCRP's wrist assembly, depicting a multi-signal coupler at the end of the arm. This coupler interface enables the rapid attachment and detachment of a primary end-effector module, accommodating mechanical, electrical, and fluid connections in a single coupling unit.

FIGS. 4A and 4B show an example of integrating different support base types for the platform in a medical application. FIG. 4A is a perspective view illustrating how a fixed (immobile) support base component can be coupled onto a mobile base. It demonstrates the platform's ability to accept an immobile column or frame as an attachment to the mobile unit, effectively combining stability with mobility. FIG. 4B is a side view of the robotic platform configured for a medical imaging procedure after such base integration. In this scenario, the platform carries a ring-shaped imaging device positioned around a patient on a support surface, exemplifying the HCRP's use in scanning or diagnostic imaging. The figure highlights how the merged base configuration allows the imaging end-effector (the ring) to be precisely positioned relative to the patient.

FIG. 5 is a perspective view of the robotic platform equipped with an alternative primary end-effector module for an industrial task. This figure depicts the HCRP reconfigured with a tool such as a heavy-duty gripper (in place of the medical/imaging modules) attached to the arm via the quick-connect coupler. It demonstrates the versatility of the platform in adapting to non-medical applications, for example gripping and manipulating an object in a manufacturing or logistics environment.

FIGS. 6A and 6B depict the attachment and removal of a ring-shaped medical device as an end-effector on the platform. FIG. 6A shows a mobile robotic platform carrying an annular imaging device (such as an MRI or CT ring) mounted as a primary end-effector on the manipulator arm. This view illustrates the platform configured to encircle or scan a subject with the ring device. FIG. 6B is an exploded perspective view illustrating the tool exchange process for the ring device. In FIG. 6B, the ring-shaped device is shown in the act of being detached from the arm's coupler, highlighting the alignment of the coupler halves and the platform's ability to automatically disconnect and swap large end-effector tools.

FIGS. 7A and 7B are schematic diagrams showing the modular structure of the platform in different base configurations. FIG. 7A is a block diagram of the HCRP with a mobile base, depicting the interconnection between the base, the robotic arm, and a sequential chain of primary, secondary, and tertiary end-effector modules. The diagram labels the coupler “connectors” at each interface, illustrating how power and control signals pass through each quick-connect joint. FIG. 7B is a similar schematic for a platform with an immobile (fixed) base, likewise showing the robotic arm mounted on a stationary support and the series of detachable end-effectors. These diagrams emphasize the common architecture of coupler-linked modules in both mobile and fixed versions of the system.

FIG. 8 illustrates examples of tertiary end-effectors that can be interchangeably attached to the platform. This figure provides a perspective view of several representative tools at the tertiary level, such as a medical instrument, a sensor unit, or other specialized implements, which can be mounted at the end of a secondary module. The arrangement demonstrates the platform's capability to host multiple kinds of task-specific tools as the final end-effector in the chain.

FIGS. 9A and 9B show the robotic platform in two different operational modes as part of a medical procedure sequence. FIG. 9A is a perspective view of the platform configured for a diagnostic imaging task: the manipulator arm positions a ring-shaped imaging end-effector around a patient lying on a support, illustrating a non-invasive scanning operation. FIG. 9B is a perspective view of the same platform reconfigured for a subsequent therapeutic or surgical task on the patient. In this view, the imaging ring has been swapped out for a different tertiary tool (for example, a surgical instrument or treatment device), demonstrating the HCRP's ability to transition between imaging and intervention by changing end-effectors.

FIGS. 10A and 10B depict the modular reconfiguration process of the platform with a series of different attachments. FIG. 10A presents multiple views of the HCRP (with a mobile base) outfitted in three example configurations: in one view, the platform is shown carrying a patient support module (such as a stretcher or bed frame); in another, it is equipped with a ring-shaped imaging module; and in a further view, it includes a linear extension module as an end-effector. These examples illustrate how the base platform can be quickly adapted for varied functions. FIG. 10B provides corresponding exploded views for each configuration from FIG. 10A, focusing on the coupler interfaces. In FIG. 10B, each end-effector module (the patient support, the imaging ring, and the extension module) is shown aligned with the robotic arm's wrist coupler prior to attachment. This demonstrates the quick-connect mechanism that enables each module to be attached or removed, highlighting the alignment features and connectors that ensure seamless module swaps.

FIGS. 11A and 11B illustrate a cooperative operation between two robotic platforms for transferring a workpiece (in this case, a patient on a support) from one platform to another. FIG. 11A is a perspective view showing two HCRP units working in tandem to hand off a patient bed or stretcher. One platform is positioned at the head end and the other at the foot end of the support, coordinating their manipulator arms to safely move the patient. FIG. 11B is a schematic sequence diagram (top or side view) of the transfer operation, marked with positions P1, P2, and P3. These sequential positions indicate the stages of the hand-off: starting alignment (P1), mid-transfer (P2), and completion with the second platform fully supporting the patient (P3). Together, FIGS. 11A and 11B demonstrate the method by which multiple robotic platforms can synchronize their actions to perform a cooperative task.

FIGS. 12A and 12B show the robotic platform engaged in an emergency or patient-handling scenario, further highlighting multi-platform cooperation. FIG. 12A is a perspective view of an HCRP unit assisting in lifting or carrying a person on a backboard or stretcher. In this view, the robotic arm is securing the person onto a support board, illustrating how the platform can function as a robotic medical assistant or “nurse” to safely handle a patient. FIG. 12B is another perspective view illustrating the delivery of that supported patient into a fixed device or station. Here, a mobile HCRP is positioning the stretcher (with the patient) into alignment with a stationary unit containing a ring-shaped device (for example, an imaging or treatment gantry). This figure demonstrates the platform's ability to interface with stationary equipment, showing a coordinated hand-off where the mobile robot inserts the patient-carrying stretcher into the fixed machine for further care or analysis.

FIG. 13 is a diagrammatic view of multiple hybrid robotic platforms deployed in a shared working environment under coordinated control. This figure depicts several HCRP units performing distinct tasks in unison-for instance, one platform might be handling a workpiece while another conducts an inspection-all managed by a central control system. The illustration conveys how the platforms communicate and synchronize their actions, highlighting features like the sharing of tool resources or passing of objects between robots. It exemplifies the broader system-level operation in which a fleet of such robotic platforms can collaborate to accomplish complex workflows.

FIG. 14 illustrates various alternative locomotion configurations for the robotic platform's support base. Shown in this figure are examples including a wheeled base, a tracked base, a legged walking base, and an aerial drone-type base. These examples demonstrate the versatility of the platform's base, indicating that the core robotic arm and modular tool system can be mounted on different types of mobility platforms (terrestrial or aerial) depending on the operational environment. FIG. 14 highlights that the HCRP concept encompasses adaptability to diverse terrain and movement requirements, from ground-based mobility (wheels or tracks) to walking mechanisms or flight-enabled bases.

FIG. 15 depicts an exemplary multi-step operation carried out by the robotic platform using different end-effectors in succession. In this figure, a single HCRP unit is shown performing a series of distinct tasks on a workpiece or within an environment by sequentially swapping its tools. For example, the platform may first use a manipulator or gripper tool to pick up an object, then exchange that for a processing tool (such as a cutting or welding tool) to perform an operation on the object, and finally switch to an inspection device (such as a camera or scanner) to evaluate the result. FIG. 15 illustrates the platform's ability to seamlessly transition between these tasks, underscoring the efficiency of using one reconfigurable robot to handle an entire sequence that traditionally might require multiple specialized machines.

FIG. 16 is an exploded detailed view of the multi-signal coupler assembly utilized in the platform. This figure breaks out the components of the coupler interface that joins an end-effector module to the robotic arm. It shows the mechanical latching mechanism, alignment guides, electrical contact pads, and fluidic connectors in a separated arrangement, allowing each feature to be identified. FIG. 16 thus highlights how the coupler provides simultaneous rigid attachment, power/data connection, and fluid line coupling between the arm and a tool. This detailed view emphasizes the engineered elements that enable quick, reliable tool exchanges with proper alignment and connectivity for both power and control signals as well as any pneumatic or hydraulic lines.

FIG. 17 is a flow diagram showing the method of automated task execution using the platform comprising receiving a task specification, commanding an intelligent management unit, retrieving and coupling a primary end-effector, a secondary end-effector, and the tertiary end-effector or the workpiece in sequence, compensating base and manipulator motions for shifting mass distribution, performing the task at the work location, and upon a task completion, returning the primary end-effector, the secondary end-effector, and the tertiary end-effector or the workpiece to respective docking bays.

### DETAILED DESCRIPTION OF THE INVENTION

The detailed embodiments of the present invention are disclosed herein. The disclosed embodiments are merely exemplary of the invention, which may be embodied in various forms. The details disclosed herein are not to be interpreted as limiting, but merely as the basis for the claims and as a basis for teaching one skilled in the art how to make and use the invention.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Throughout this specification, the word “comprise,” or variations thereof such as “comprises” or “comprising,” will be understood to imply the inclusion of a stated element, integer or step, or group of elements integers or steps, but not the exclusion of any other element, integer or step, or group of elements, integers or steps.

Index of Labelled Features in Figures. Features are listed in numeric order. Referring to the Figures, the following features are shown:

Element 100 which is a hybrid compounded robotic platform.

Element 110 which is a support architecture.

Element 120 which is a mobile support base.

Element 130 which is an immobile support base.

Element 140 which is a detachable combination.

Element 150 which is a robotic manipulator arm.

Element 160 which is a wrist assembly.

Element 170 which is a primary end-effector.

Element 180 which is a workpiece.

Element 190 which is a first multi-signal coupler.

Element 200 which is a secondary end-effector.

Element 210 which is a second multi-signal coupler.

Element 220 which is a tertiary end-effector.

Element 230 which is a third multi-signal coupler.

Element 240 which is an actuator.

Element 250 which is a parking station.

Element 260 which is a docking seat.

Element 270 which is an alignment fixture.

Element 280 which is an electrical contact.

Element 290 which is an intelligent management unit.

Element 300 which is a received task instruction.

Element 310 which is a balance sensor.

Element 320 which is a collision-avoidance sensor.

Element 330 which is a power module.

Element 340 which is an interlocking mechanical latch.

Element 350 which is a plurality of electrical power and data contacts.

Element 360 which is a fluidic conduit.

Element 370 which is a dedicated drive motor.

Element 380 which is a medical device.

Element 420 which is an inspection or imaging device.

Element 430 which is a collaborative robot arm.

Element 440 which is an individualized docking bay.

Element 450 which is a passive alignment pin.

Element 460 which is a spring-loaded electrical contact.

Element 480 which is a coupling interface.

Element 490 which is a combined base.

Element 500 which is an elbow assembly.

Element 510 which is a lifting column.

Element 520 which is an autonomous battery.

Element 530 which is an on-board power source.

Element 540 which is a base-to-base multi-signal connector.

Element 550 which is a system of robotic platforms.

Element 560 which is a plurality of platforms.

Element 570 which is a central coordination server.

Element 580 which is a first platform.

Element 590 which is a second platform.

Element 600 which is an end-effector or workpiece exchange across multiple bases.

Element 610 which is a dynamic map. (Not shown.)

Element 620 which is a support architecture state of each of the plurality of platforms

Element 630 which is a parking-station inventory.

Element 640 which is a tool identity.

Element 660 which is a specified secondary end-effector.

Element 670 which is a method of automated task execution.

Element 680 which is a work location.

Element 690 which is a task.

Element 700 which is a multi-signal coupler.

Element 710 which is a receipt of the task specification.

Element 720 which is a voice command.

Element 730 which is a structured task plan.

Element 740 which is an inter-platform exchange.

Element 750 which is an alignment verification.

Element 760 which is a coupler engagement.

Element 770 which is an inventory update.

The following detailed description sets forth exemplary embodiments of the hybrid compounded robotic platform in sufficient detail to enable a person skilled in the art to make and use the invention. It is to be understood that variations in form and detail may be made without departing from the spirit and scope of the invention. The invention is not limited to the specific examples described herein but may be embodied in various forms.

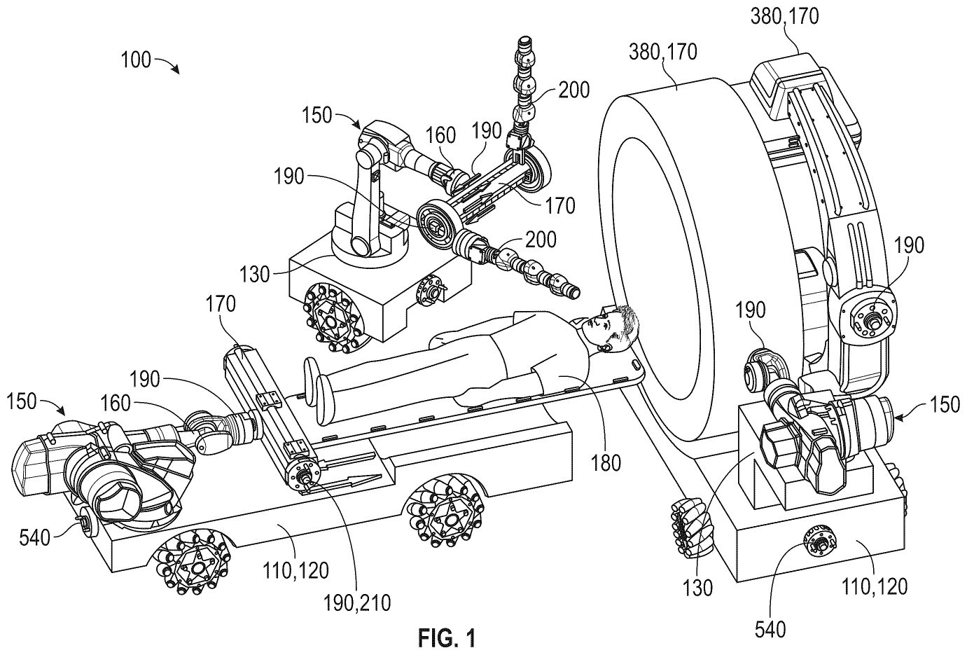

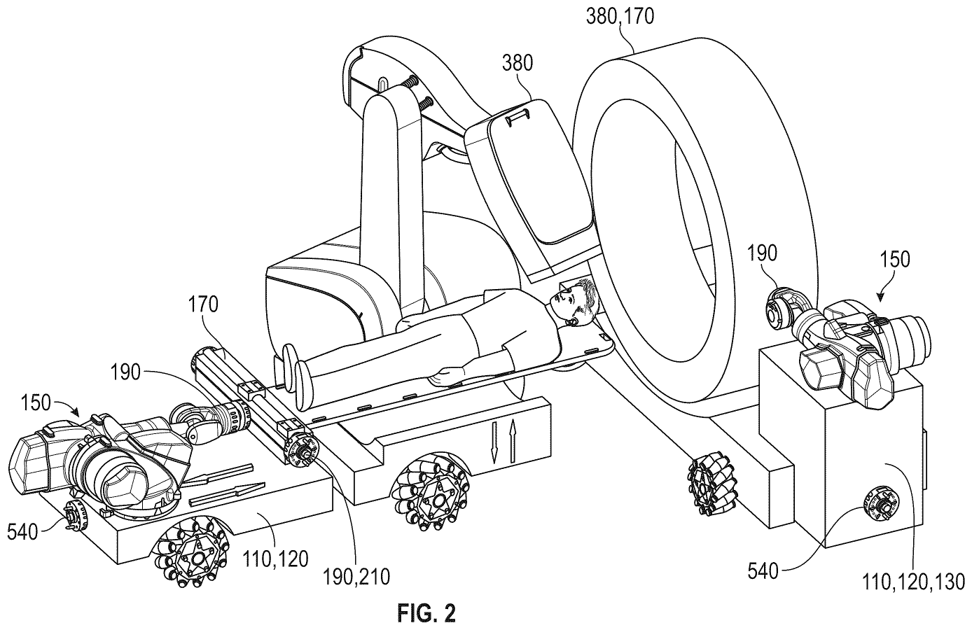

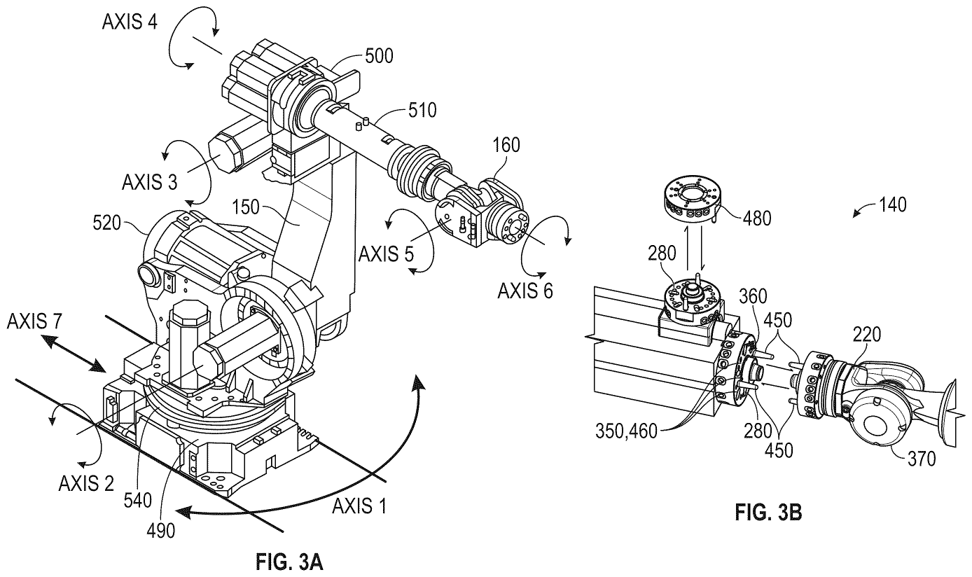

Referring to FIG. 1, the hybrid compounded robotic platform includes a support base which may be constructed as either a mobile or an immobile structure. In mobile embodiments, the base may include omni-directional wheels, caterpillar tracks, walking legs, or aerial/underwater propulsion systems. In immobile embodiments, the base may be a fixed column, lifting column, or rail-mounted carriage. The base houses internal power supply units, battery systems, control electronics, and connection points for the parking station and intelligent management unit. The base is manufactured using rigid, load-bearing materials such as aluminum alloy, carbon-fiber composites, or high-strength steel, selected to support the weight and dynamic forces of the manipulator arm and attached end-effectors.

Mounted to the support base is a robotic manipulator arm terminating in a wrist assembly. The manipulator arm may be a six-axis industrial arm, a seven-degree-of-freedom collaborative arm, or a custom actuator array, depending on the application. The joints are powered by servo motors, harmonic drives, or hydraulic actuators, each with integrated encoders for precise position feedback. The wrist assembly is adapted to interface with a primary multi-signal coupler, allowing rapid attachment and removal of the primary end-effector. The manipulator arm is constructed with internal cable routing channels to protect and organize electrical, data, and fluid lines leading to the coupler.

The primary multi-signal coupler is a precision-engineered interface that mates with a corresponding coupler half mounted on the primary end-effector. It is fabricated from lightweight, high-strength alloys with corrosion-resistant finishes. The coupler includes: an interlocking mechanical latch with spring-loaded retention; electrical contacts rated for both power and high-speed data transmission; and fluidic conduits with quick-seal valves for pneumatic, hydraulic, or coolant transfer. The coupler is designed for automatic alignment and engagement under manipulator control, with alignment pins and tapered guide surfaces to ensure correct mating.

The primary end-effector may be manufactured in different forms depending on intended use, such as a linear guide system, a heavy-duty gripper, or a robotic sub-arm. It carries its own actuator(s) —such as servo motors, pneumatic cylinders, or hydraulic pistons —to independently drive the secondary end-effector. The housing of the primary end-effector is machined or 3D-printed from lightweight materials to minimize load on the manipulator arm while ensuring rigidity. The embedded control electronics interface with the intelligent management unit via the coupler's electrical contacts.

The secondary end-effector is mounted to the primary end-effector via a secondary multi-signal coupler of similar design to coupler. It also carries its own actuator(s) to independently position and operate the tertiary end-effector. The independent actuation of each stage allows for complex, multi-point manipulation without moving the main manipulator arm, improving precision and efficiency.

The tertiary end-effector, mounted via a tertiary multi-signal coupler, may be any specialized tool, such as a medical imaging device, a precision gripper, or a collaborative micro-arm. The tertiary tool is designed for direct interaction with the workpiece or environment and may include integrated sensors for vision, force feedback, or environmental measurement.

The parking station is either integrated into or mounted on the support base. It is fabricated from rigid structural materials and contains docking bays shaped to the geometry of the primary, secondary, and tertiary end-effectors. Each bay includes passive alignment pins and electrical contact arrays for charging tool batteries and performing calibration or self-test routines. The docking bays are arranged to permit unobstructed approach by the manipulator arm for smooth coupling and decoupling operations.

The intelligent management unit is a microprocessor-or FPGA-based control system in electronic communication with the manipulator arm, multi-signal couplers, parking station, and any mounted sensors. It executes stored algorithms to: sequence tool exchanges in response to task commands; monitor power, balance, and collision-avoidance sensors; adjust manipulator movements in real time; command the parking station to charge, calibrate, or test tools upon docking. The control unit may include wired or wireless communication interfaces to receive commands locally or from a central coordination server in multi-platform deployments.

To make the platform, a skilled builder assembles the support base and mounts the manipulator arm with wrist assembly. The multi-signal couplers are installed on both the manipulator side and the tool side of each end-effector. The parking station is fixed to the base or positioned adjacent to the operating area. Control electronics for the intelligent management unit are programmed with tool identification, docking routines, and compensation algorithms.

To use the platform, an operator or automated control system issues a task command identifying the required tertiary end-effector and operation. The intelligent management unit decouples any non-required tools and docks them in their designated bays for charging or calibration. It then retrieves and attaches the necessary primary, secondary, and tertiary end-effectors in sequence, using the couplers'automatic alignment and engagement features. During task execution, the intelligent management unit compensates for any shifts in center-of-mass, monitors sensors for collision risks, and adjusts manipulator and base movements accordingly. Upon completion, the tools are returned to their docking bays, where charging and self-testing are initiated automatically.

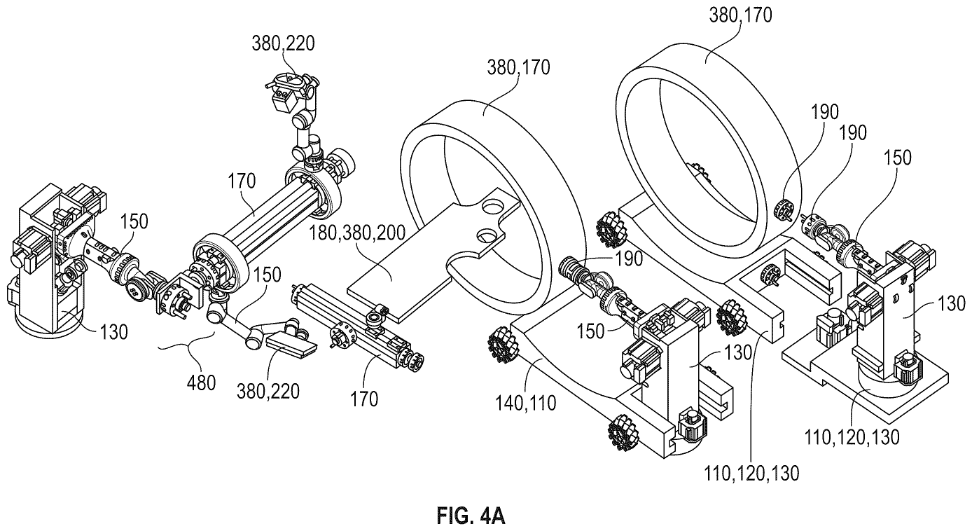

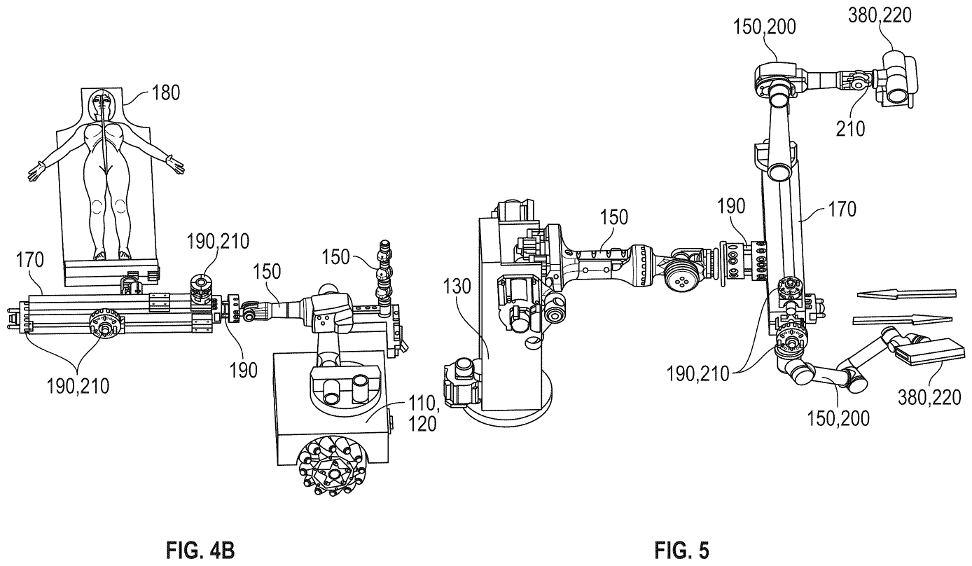

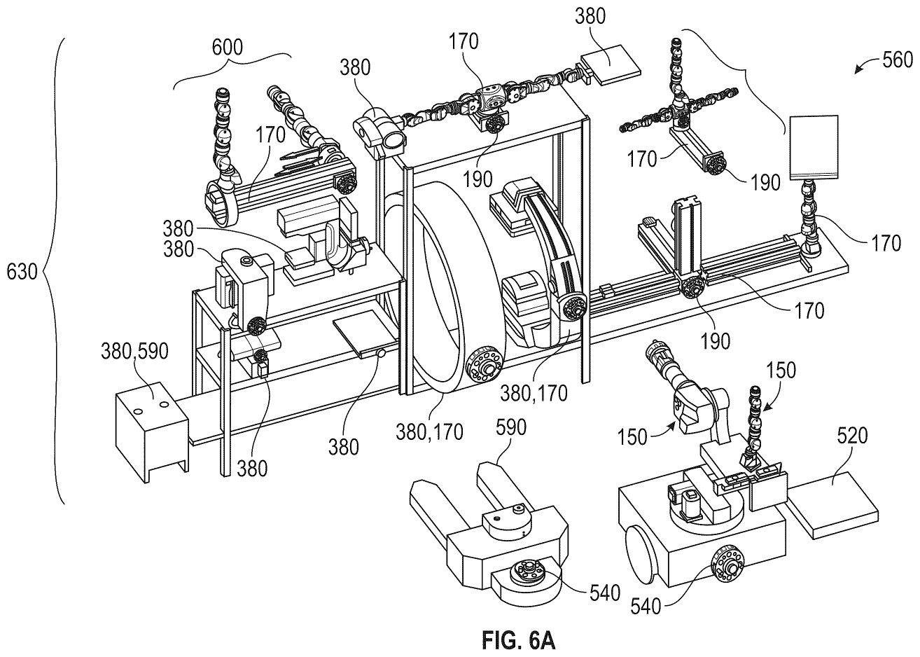

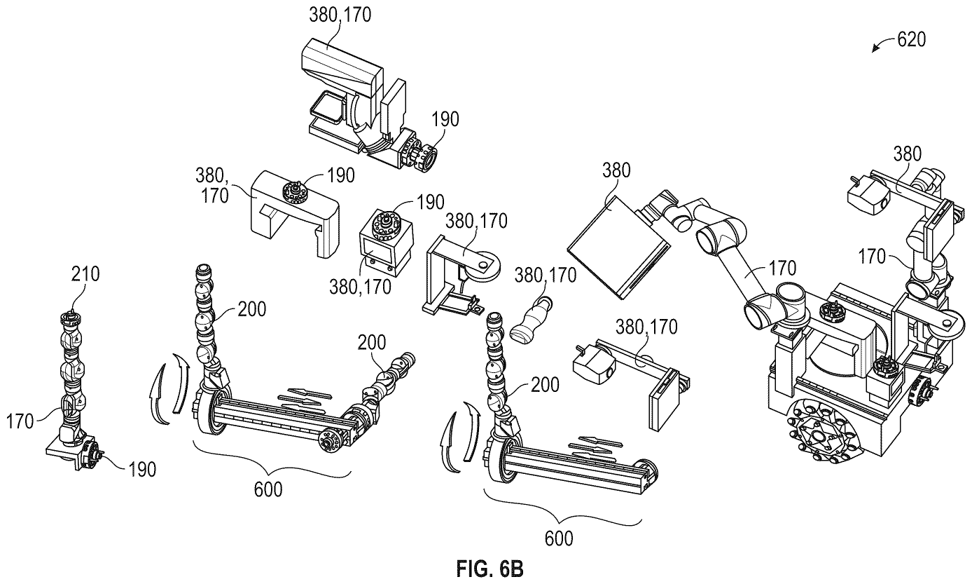

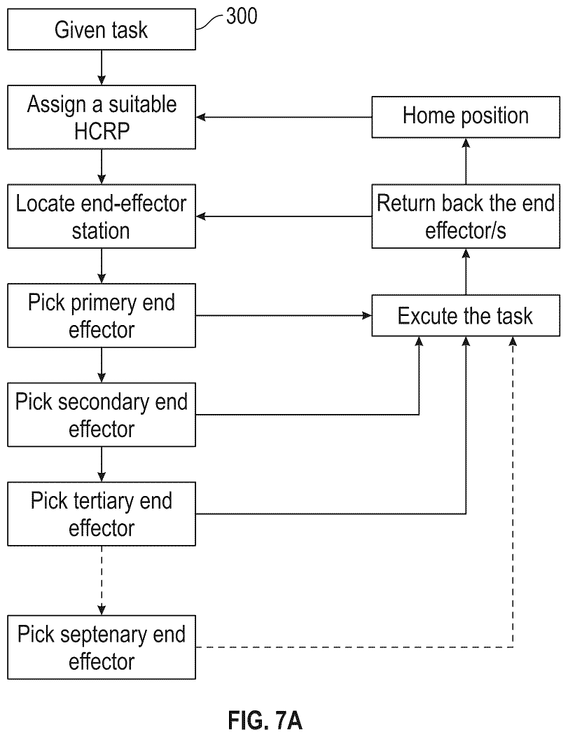

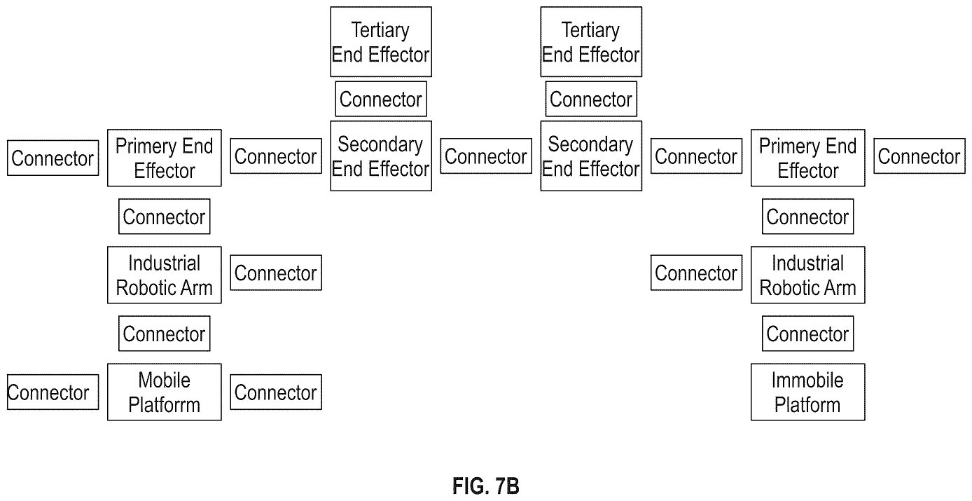

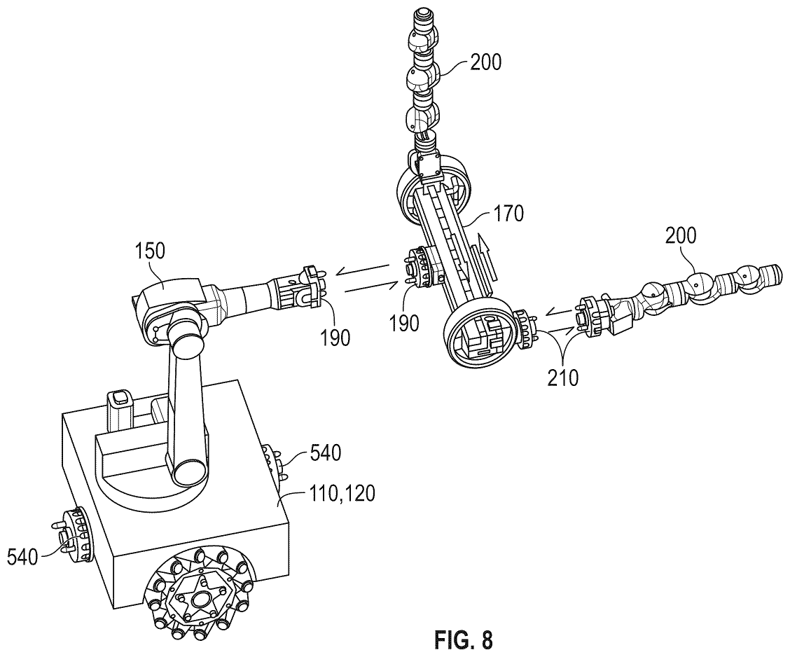

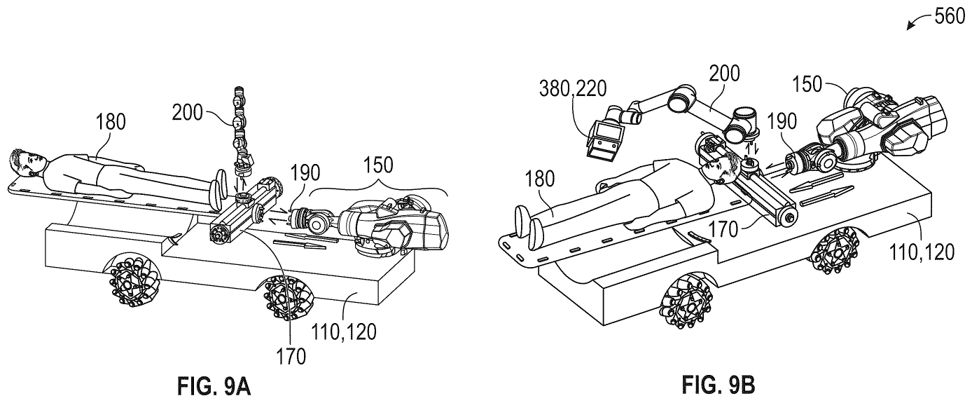

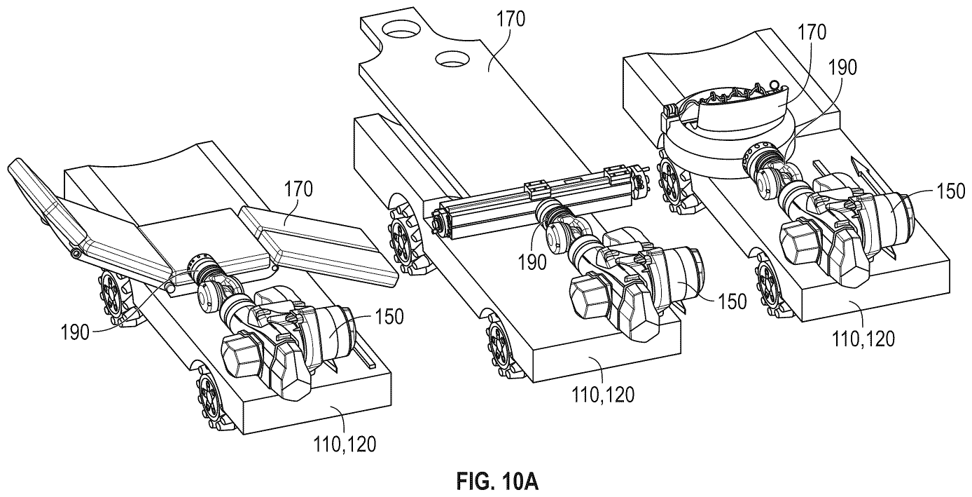

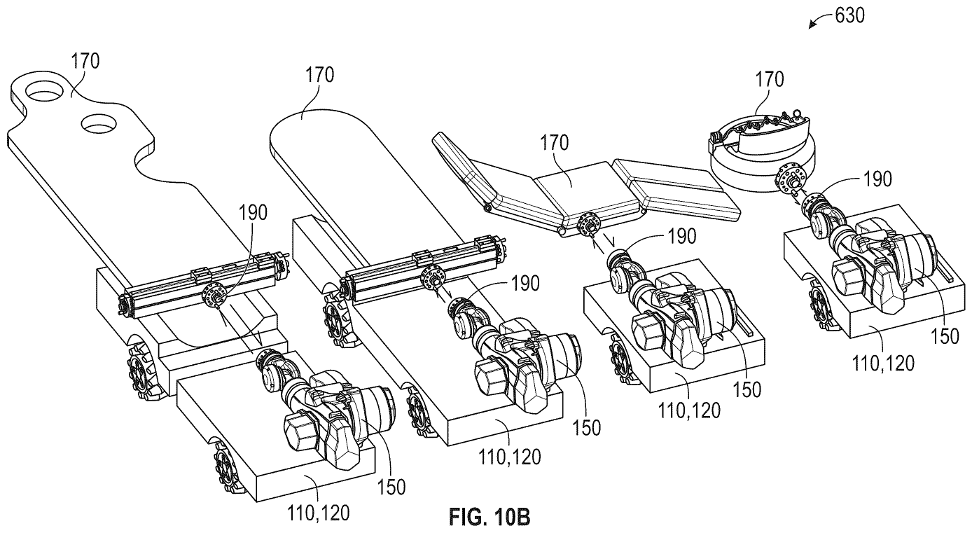

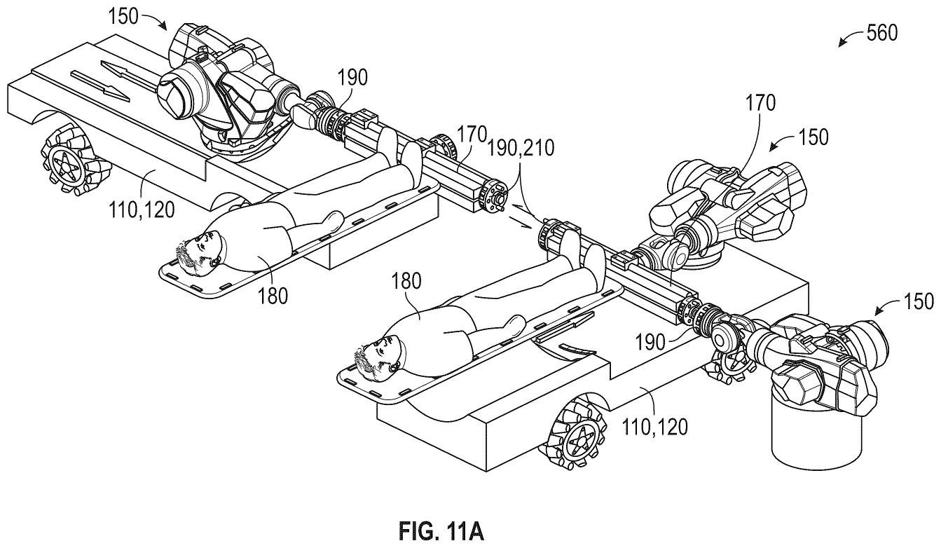

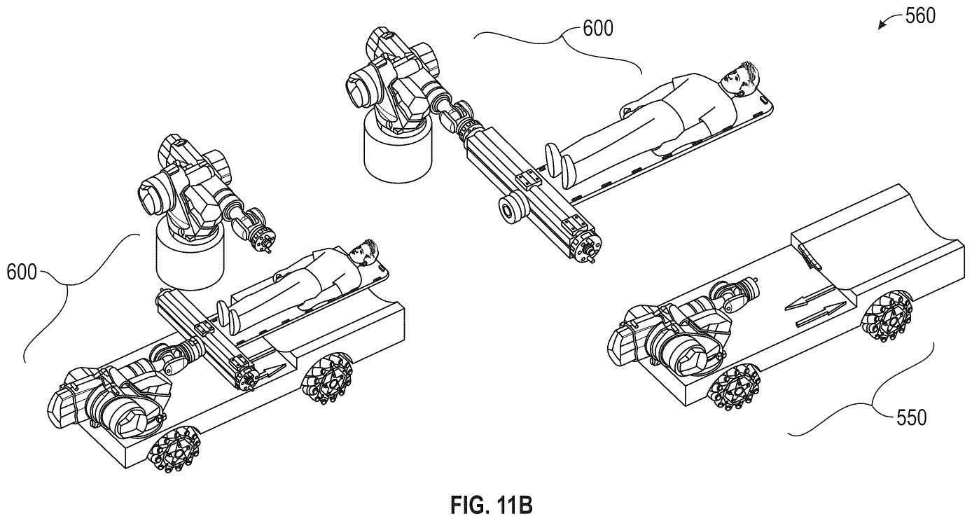

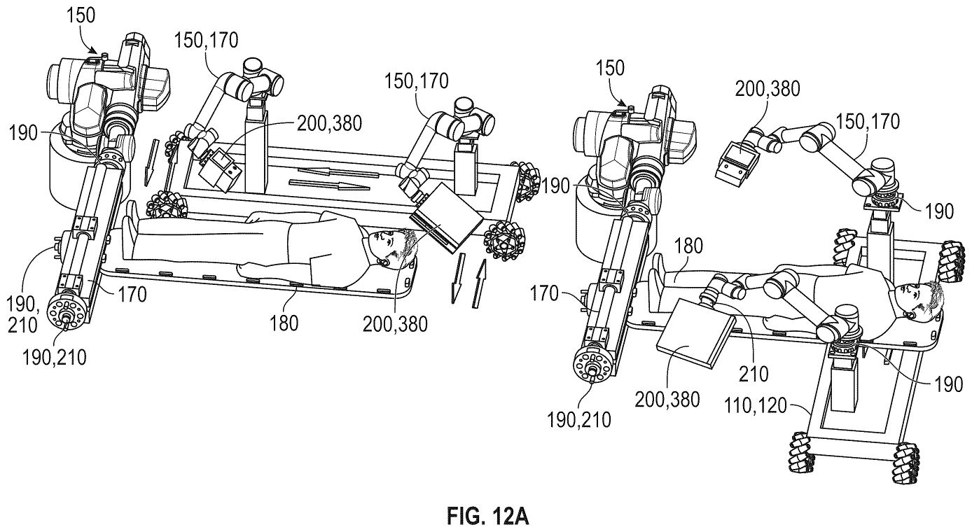

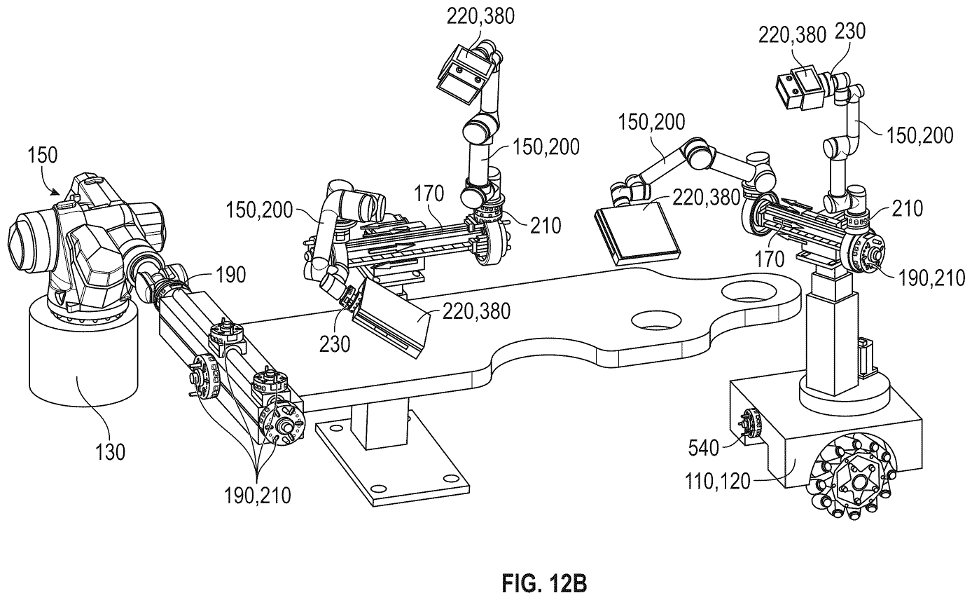

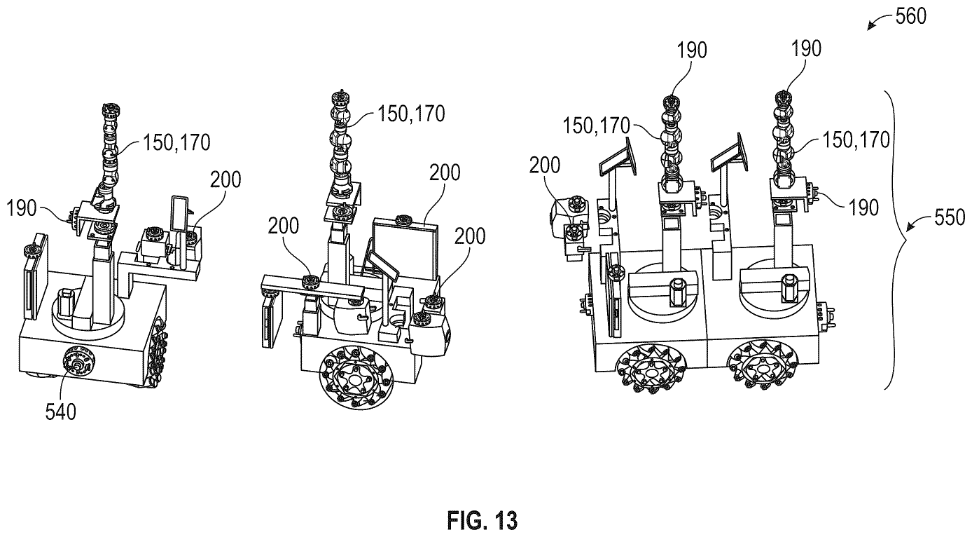

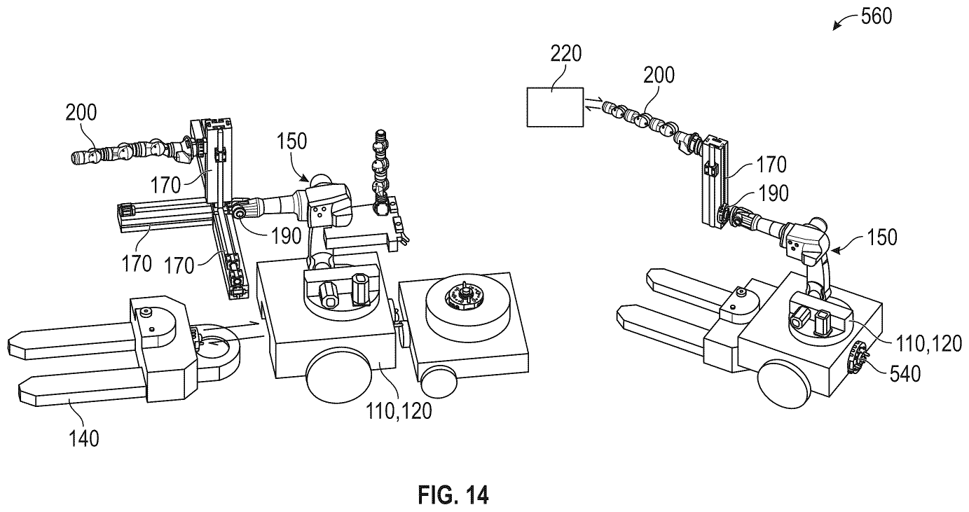

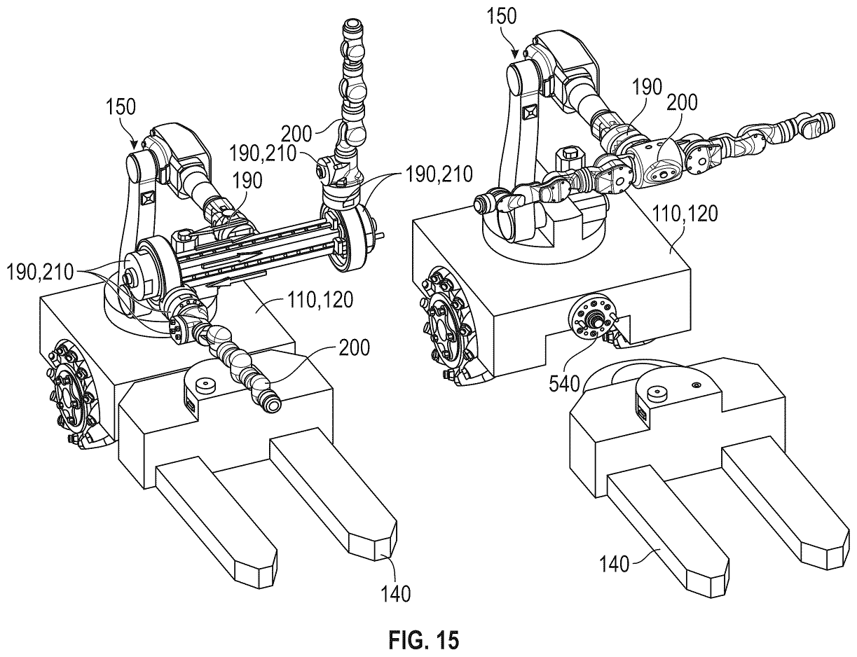

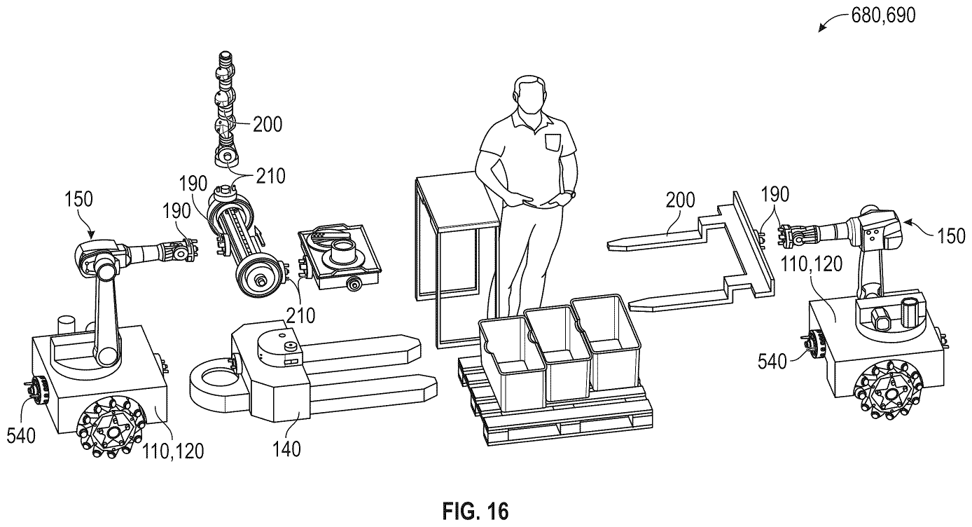

In a multi-platform configuration, as illustrated schematically in FIG. 4, multiple hybrid compounded robotic platforms are deployed within a shared operating environment. Each platform is built according to the construction described above, with its own support base, manipulator arm, sequential end-effectors, docking station, and intelligent management unit.

A central coordination server is implemented as a high-performance computing unit running task allocation and fleet management software. The server maintains continuous bidirectional communication with each intelligent management unit via a secure wired or wireless network. The physical server may be hosted locally within the facility or remotely via a cloud infrastructure, provided that communication latency remains within operational tolerances for coordinated motion control and collision avoidance.

To make the multi-platform system, the builder installs networking hardware, configures IP addressing or other device identifiers for each platform, and loads the coordination software onto the central server. Each intelligent management uniti s programmed to share its status, position, and available tools with the central server. A dynamic map of the environment, including the real-time location of each support base and the status of each docking bay, is maintained by the server in memory.

To use the multi-platform system, the central coordination server receives a task list and analyzes it to determine which platforms are best suited for each job based on current location, available end-effectors, and battery charge status. The server may instruct a first platform to deliver a specified secondary end-effector to a second platform's docking bay. This transfer is executed by the intelligent management unit of the first platform, which retrieves the requested tool from its own docking station, navigates to the second platform's location, and couples the tool into the receiving docking bay using passive alignment features and electrical contacts. The server updates its map to reflect the transfer in real time.

In certain embodiments, the coordination server resolves inter-platform collision risks by broadcasting position and trajectory updates to all platforms within range, allowing each intelligent management unit to adjust its navigation path and manipulator motions to prevent interference. This shared data environment enables complex cooperative operations such as synchronized assembly, multi-robot lifting, or sequential inspection along a production line.

The invention also includes a method of automated task execution implemented by the intelligent management unit, either operating independently or under coordination server supervision. To make the method executable, the management unit firmware is programmed with a structured sequence of operations including end-effector identification, docking/undocking control, motion compensation algorithms, and task-specific execution routines.

To use the method, an operator or higher-level control system first provides a task specification that identifies the required tertiary end-effector and work location. This specification may be entered manually via a console, transmitted as a digital job file, or spoken as a voice command that is parsed by onboard voice recognition software.

Upon receiving the task specification, the intelligent management unit executes the following steps:
Step 1—Tool Preparation: Any end-effectors not required for the task are decoupled from the manipulator arm and returned to their designated docking bays. Upon docking, electrical contacts initiate charging of internal tool batteries and run self-calibration routines.
Step 2—Tool Retrieval: The manipulator arm, under control of the intelligent management unit, retrieves the required primary, secondary, and tertiary end-effectors in sequence, engaging each via its respective multi-signal coupler. Alignment pins and tapered guides ensure correct orientation before latching.
Step 3—Motion Compensation: As each tool is attached, the management unit adjusts manipulator joint torques and base drive parameters to compensate for changes in the center of mass, ensuring stability during movement.
Step 4—Task Execution: The platform navigates to the work location using onboard or external guidance, performs the programmed task with the tertiary end-effector, and monitors real-time feedback from sensors to adapt motions for precision and safety.
Step 5—Post-Task Servicing: Upon task completion, the manipulator arm 110 returns all tools to their docking bays, where automated charging and post-use calibration occur. Status logs are updated and transmitted to the coordination server 300 for record-keeping and fleet management.

In embodiments where voice command input is enabled, the system includes a microphone array and natural language processing software. Spoken instructions such as “Inspect module three with imaging tool” are converted into structured task plans specifying the correct tertiary end-effector, work location, and sequence of operations. These plans are forwarded wirelessly to the intelligent management unit for execution without requiring manual programming.

The integration of the above hardware and software elements enables a single hybrid compounded robotic platform—or a fleet of such platforms—to perform a wide range of tasks with minimal downtime, rapid tool changes, and optimized allocation of shared resources. The described system and method provide complete functional coverage for the claim set while enabling practical, real-world deployment in industries where adaptability, speed, and coordinated multi-robot operations are essential.

In one option, the support base is configured as a low-profile autonomous mobile robot equipped with omni-directional mecanum wheels and integrated LIDAR for precise indoor navigation. This embodiment is particularly suited for environments such as hospitals, factories, or warehouses where maneuverability in confined spaces is critical. The mobile base may further include an active suspension system to maintain stability when carrying heavy or asymmetrically loaded end-effectors.

In another option, the support base is immobile, configured as a floor-mounted lifting column with a linear guide rail to vertically position the manipulator arm. This arrangement is optimized for high-precision assembly stations or medical operating theaters where stability and positional repeatability are paramount.

The manipulator arm may be implemented in several configurations. In one option, the arm is an industrial six-axis assembly capable of high payload capacity for manufacturing and construction tasks. In another option, the arm is a lightweight seven-degree-of-freedom collaborative manipulator designed for safe operation around human workers, with integrated torque sensing for collision detection and compliant motion.

The primary, secondary, and tertiary end-effectors can be configured for specific industries. In a medical application, the primary end-effector may be a robotic positioning stage, the secondary a fine-movement surgical manipulator, and the tertiary a specialized imaging or cutting tool. In a logistics setting, the primary may be a telescoping conveyor, the secondary a robotic gripper, and the tertiary a barcode scanner or RFID reader.

The multi-signal couplers can be adapted for different operating environments. For cleanroom applications, the couplers may be sealed against particulate ingress and rated for sterilization processes. For harsh outdoor or underwater use, they may be fabricated from marine-grade stainless steel with corrosion-resistant seals and pressure-rated conduit connections.

The parking station can be implemented in multiple layouts. In compact environments, a vertical docking tower may be integrated into the base to save floor space. In large-scale industrial deployments, docking bays may be positioned along a wall or production line, allowing multiple platforms to share tool resources without crowding.

The intelligent management unit may vary in computational capability. For single-platform standalone use, it may consist of an embedded processor with real-time control firmware. In high-throughput multi-platform systems, the unit may include an AI-enabled processor capable of machine learning-based motion optimization and predictive maintenance scheduling.

Optional sensor arrays 210 may include high-resolution 3D cameras, thermal imagers, ultrasonic range finders, or chemical detectors, enabling the platform to adapt to specialized tasks such as structural inspection, environmental monitoring, or hazardous material handling.

The present invention delivers a unique combination of adaptability, modularity, and scalability not available in existing robotic platforms. By enabling rapid exchange of primary, secondary, and tertiary end-effectors via multi-signal couplers, the platform consolidates multiple specialized robotic functions into one reconfigurable system.

The integrated parking station not only stores tools securely but also charges and calibrates them automatically, reducing downtime and ensuring that each tool is always ready for use. This feature enhances operational readiness across industries where tool performance is mission-critical, such as surgical theaters, production lines, or field repair operations.

The intelligent management unit allows real-time control of both individual and multi-platform configurations. This enables optimized task sequencing, collision avoidance, and automatic tool sharing between units, which directly reduces labor overhead and increases throughput.

The invention's ability to operate with mobile, immobile, or hybrid support bases allows deployment in environments ranging from confined indoor spaces to rugged outdoor terrains. This versatility eliminates the need for separate, environment-specific robots, cutting procurement and maintenance costs.

Multi-platform coordination via the central server 300 creates a networked robotic ecosystem capable of handling large-scale, multi-step processes more efficiently than isolated units. Tool-sharing capability prevents redundancy in tool inventories and allows task specialization without requiring every unit to carry every tool.

The system's ability to compensate for center-of-mass changes during tool exchanges ensures stability and safety, even when manipulating heavy or unevenly balanced loads. This feature enables reliable operation in sensitive environments such as cleanrooms, laboratories, and human-occupied spaces.

The voice-command and high-level instruction interface reduces the technical barrier for human operators, allowing rapid task assignment without specialized programming knowledge. This significantly expands the potential user base and accelerates task initiation.

Collectively, these advantages enable the present invention to replace multiple single-purpose robotic systems with a single, flexible, and future-proof platform. The result is a reduction in capital and operational costs, an increase in adaptability to changing requirements, and the ability to perform a broader range of tasks with fewer physical assets.

In a preferred embodiment of the invention, there is a hybrid compounded robotic platform comprising: a support architecture including a mobile support base, an immobile support base, or a detachable combination thereof; at least one robotic manipulator arm mounted to said support architecture and terminating in a wrist assembly; a primary end-effector or a workpiece detachably coupled to said wrist assembly via a first multi-signal coupler that provides mechanical, electrical, and fluidic connections; a secondary end-effector detachably coupled to said primary end-effector via a second multi-signal coupler; a tertiary end-effector detachably coupled to said secondary end-effector via a third multi-signal coupler; wherein said primary end-effector includes actuators configured to drive movement of said secondary end-effector, and said secondary end-effector includes actuators configured to drive movement of said tertiary end-effector; a parking station integrated into or on said support architecture or implemented as an external station, said parking station including shaped docking seats and alignment fixtures matching each of said primary end-effector, said secondary end-effector, and said tertiary end-effector or said workpiece, and further including electrical contacts for automatic charging and calibration when docked; and an intelligent management unit in communication with said robotic manipulator arm, said multi-signal coupler, and said parking station, said intelligent management unit programmed to automatically sequence coupling and decoupling of said primary end-effector, said secondary end-effector, and said tertiary end-effector or said workpiece based on a received task instruction, a monitor power, a balance sensor, and a collision-avoidance sensor and adjust said robotic manipulator arm and said mobile support base motions in real time as said primary end-effector, said secondary end-effector, and said tertiary end-effector or said workpiece are exchanged, and command said parking station to charge, calibrate, self-test, or swap a power module for any docked end-effector or said workpiece.

In an alternate embodiment of the invention, there is a hybrid compounded robotic platform wherein said first multi-signal coupler, said second multi-signal coupler, or said third multi-signal coupler comprises an interlocking mechanical latch, a plurality of electrical power and data contacts, and one or more fluidic conduits, each arranged to automatically mate when halves of said multi-signal coupler engage, with tapered guides and alignment pins to ensure a proper orientation.

In an alternate embodiment of the invention, there is a hybrid compounded robotic platform wherein each of said primary end-effector, said secondary end-effector, and said tertiary end-effector in a chain includes at least one jointed degree of freedom that is rotational or prismatic, and a dedicated drive motor, such that said dedicated drive motor of said primary end-effector drives a positioning of said secondary end-effector in at least one axis independent of said robotic manipulator arm.

In an alternate embodiment of the invention, there is a hybrid compounded robotic platform wherein said primary end-effector, said secondary end-effector, or said tertiary end-effector comprises a medical device, a manufacturing tool, a logistics tool, a construction tool, an inspection or imaging device, or a collaborative robot arm.

In an alternate embodiment of the invention, there is a hybrid compounded robotic platform wherein said parking station comprises three individualized docking bays, each shaped to a geometry of one of said primary end-effector, said secondary end-effector, and said tertiary end-effector or said workpiece, and further comprising passive alignment pins and spring-loaded electrical contacts for auto-alignment and reliable electrical engagement.

In an alternate embodiment of the invention, there is a hybrid compounded robotic platform wherein said intelligent management unit implements a control algorithm that compensates for shifts in center-of-mass of said robotic manipulator arm as said primary end-effector, said secondary end-effector, and said tertiary end-effector or said workpiece are exchanged, by adjusting base drive and joint torques in real time based on tool identity and mass properties.

In an alternate embodiment of the invention, there is a hybrid compounded robotic platform wherein said support architecture includes a coupling interface by which said immobile support base can be detachably mounted on said mobile support base to form a combined base for task execution and then detached for redeployment.

In an alternate embodiment of the invention, there is a hybrid compounded robotic platform wherein said robotic manipulator arm is forearm-only, including said wrist assembly and an elbow assembly projected from said support architecture, and omitting a shoulder and an upper arm, or wherein said robotic manipulator arm is configured with said elbow assembly directly connected to said support architecture.

In an alternate embodiment of the invention, there is a hybrid compounded robotic platform wherein at least one collaborative robot arm is configured as said primary end-effector detachably coupled to said wrist assembly or to a lifting column mounted on said support architecture, and wherein one or more secondary end-effectors are detachably mounted to said collaborative robot arm.

In an alternate embodiment of the invention, there is a hybrid compounded robotic platform wherein said parking station is mounted on said robotic manipulator arm or said wrist assembly to shorten tool travel paths, or is external and networked to allow multi-platform sharing of said primary end-effector, said secondary end-effector, and said tertiary end-effector or said workpiece.

In an alternate embodiment of the invention, there is a hybrid compounded robotic platform further comprising an autonomous battery or power-module swapping at said parking station for any said primary end-effector, said secondary end-effector, or said tertiary end-effector or said workpiece that includes an on-board power source, said autonomous battery or power-module swapping triggered by said intelligent management unit based on state-of-charge thresholds.

In an alternate embodiment of the invention, there is a hybrid compounded robotic platform further comprising a base-to-base multi-signal connector enabling two or more platforms to be physically coupled at support bases to share power, data, and fluids and to perform cooperative operations as a unified system.

In a preferred embodiment of the invention, there is a system of robotic platforms comprising: a plurality of platforms according to claim 1; and a central coordination server in networked communication with an intelligent management unit of each of said plurality of platforms including at least a first platform and a second platform, said central coordination server programmed to allocate tasks, synchronize an end-effector or workpiece exchange across multiple bases, and resolve inter-platform collision risks via shared position and trajectory data.

In an alternate embodiment of the invention, there is a system of robotic platforms wherein said central coordination server maintains a dynamic map of a support architecture state of each of said plurality of platforms, a parking-station inventory, a tool identity, and a battery status, and autonomously directs a first platform to deliver a specified secondary end-effector or a workpiece to a parking station of a second platform for exchange.

In an alternate embodiment of the invention, there is a system of robotic platforms wherein said end-effector or workpiece exchange between said plurality of platforms is dynamic or static, including direct manipulator-to-manipulator transfer or parking-station mediated transfer, with verification of alignment and coupler engagement prior to release.

In an alternate embodiment of the invention, there is a system of robotic platforms wherein said central coordination server can command temporary physical coupling of two or more of said plurality of platforms via a base-to-base multi-signal connector to enable load sharing, synchronized motion, or extended reach operations.

In a preferred embodiment of the invention, there is a method of automated task execution using the platform of claim 1, comprising: receiving a task specification identifying a required tertiary end-effector or a workpiece and a work location; commanding an intelligent management unit to decouple said required tertiary end-effector or said workpiece not required for a task and dock said required tertiary end-effector or said workpiece in a parking station for charging and calibration; retrieving and coupling a primary end-effector, a secondary end-effector, and said tertiary end-effector or said workpiece in sequence via a multi-signal coupler of each of said primary end-effector, said secondary end-effector, and said tertiary end-effector; compensating base and manipulator motions for shifting mass distribution resulting from any of said primary end-effector, said secondary end-effector, or said tertiary end-effector or workpiece exchange; performing said task at said work location; and upon a task completion, returning said primary end-effector, said secondary end-effector, and said tertiary end-effector or said workpiece to respective docking bays for post-use calibration and power replenishment.

In an alternate embodiment of the invention, there is a method of automated task execution wherein a receipt of said task specification comprises parsing a voice command, converting it to a structured task plan, and forwarding said structured task plan wirelessly to said intelligent management unit.

In an alternate embodiment of the invention, there is a method of automated task execution further comprising an inter-platform exchange in which a central coordination server directs a first platform to deliver a designated secondary end-effector or said workpiece to a second platform, including an alignment verification, a coupler engagement, and an inventory update in a dynamic map.

In an alternate embodiment of the invention, there is a method of automated task execution wherein an autonomous power-module swapping is performed for a tool or said workpiece that requires replenishment, said autonomous power-module swapping is coordinated with tool calibration routines so that said tool is returned to service in a ready-to-use state.

In an alternate embodiment of the invention, there is a method of automated task execution wherein a support architecture is configured from an immobile base detachably mounted on a mobile base for said task, and is detached after completion for redeployment to a new location.

In an alternate embodiment of the invention, there is a hybrid compounded robotic platform wherein said support architecture is omni-form to accommodate and stow different shapes of said primary end-effector, said secondary end-effector, and said tertiary end-effector or said workpiece, including ring-type devices and patient-support articles, thereby enabling tool or workpiece parking and transport as part of task execution.

In a preferred embodiment of the present invention there is a hybrid compounded robotic platform comprising: a support architecture including a mobile support base, an immobile support base, or a detachable combination thereof; at least one robotic manipulator arm mounted to the support architecture and terminating in a wrist assembly; a primary end-effector or workpiece detachably coupled to the wrist assembly via a multi-signal coupler that provides mechanical, electrical, and fluidic connections; a secondary end-effector detachably coupled to the primary end-effector via a respective multi-signal coupler; and a tertiary end-effector detachably coupled to the secondary end-effector via a respective multi-signal coupler; wherein the primary end-effector includes actuators configured to drive movement of the secondary end-effector, and the secondary end-effector includes actuators configured to drive movement of the tertiary end-effector; a parking station integrated into or on the support architecture or implemented as an external station, the station including shaped docking seats and alignment fixtures matching each of the primary, secondary, and tertiary end-effectors or workpieces, and further including electrical contacts for automatic charging and calibration when docked; and an intelligent management unit in communication with the manipulator arm, the multi-signal couplers, and the parking station, the unit programmed to automatically sequence coupling and decoupling of end-effectors or workpieces based on a received task instruction, monitor power, balance, and collision-avoidance sensors and adjust manipulator and base motions in real time as end-effectors or workpieces are exchanged, and command the parking station to charge, calibrate, self-test, or swap a power module for any docked end-effector or workpiece.

This embodiment encompasses both stationary and mobile robotic configurations that can operate autonomously or collaboratively. The detachable bases allow flexible deployment between work sites, and the multi-signal couplers simplify rapid tool exchange. The intelligent management unit functions as the supervisory control layer that harmonizes motion, docking, and calibration to ensure safe, precise operation across multiple environments. Optional configurations include sealed couplers for sterile or underwater use, sensor arrays for dynamic balancing, and redundant wireless links between the base and server to prevent communication loss.

In an alternate embodiment of the present invention there is a hybrid compounded robotic platform wherein the multi-signal coupler comprises an interlocking mechanical latch, a plurality of electrical power and data contacts, and one or more fluidic conduits, each arranged to automatically mate when the coupler halves engage, with tapered guides and alignment pins to ensure proper orientation.

This configuration allows high-speed automatic connection and disconnection with minimal mechanical wear. Optional designs may employ bayonet-style latches, magnetic coupling assist, or smart contacts that confirm proper seating through impedance or signal checks. The fluidic pathways may serve pneumatic, hydraulic, or coolant functions, depending on the attached end-effector's requirements.

In an alternate embodiment of the present invention there is a hybrid compounded robotic platform wherein each end-effector in the chain includes at least one jointed degree of freedom that is rotational or prismatic, and a dedicated drive motor, such that the primary end-effector motor drives the positioning of the secondary end-effector in at least one axis independent of the manipulator arm.

This embodiment provides nested actuation—each module contributing its own movement capabilities. Optional variations include servo-driven joints for precision alignment or pneumatic cylinders for lightweight, high-speed actuation. The modular drive architecture increases dexterity while minimizing base arm load, enabling the platform to perform compound tasks in confined or intricate workspaces.

In an alternate embodiment of the present invention there is a hybrid compounded robotic platform wherein the tertiary end-effector comprises a medical device, a manufacturing tool, a logistics tool, a construction tool, an inspection or imaging device, or a collaborative robot arm.

This embodiment extends the platform's reach across sectors. For medical use, the tertiary module may host imaging rings, surgical instruments, or patient-handling devices. In industrial contexts it can mount grippers, welding heads, or inspection probes. Optional interfaces allow tertiary modules to include embedded processors for local control and quick-release features for sterile or hazardous-environment replacement.

In an alternate embodiment of the present invention there is a hybrid compounded robotic platform wherein the parking station comprises three individualized docking bays, each shaped to the geometry of one of the primary, secondary, or tertiary end-effectors or workpieces, and further comprising passive alignment pins and spring-loaded electrical contacts for auto-alignment and reliable electrical engagement.

This arrangement ensures safe, repeatable storage and retrieval of modules. The docking station may include environmental controls such as temperature regulation, UV sterilization, or dust sealing. Optional sensors in each bay verify docking integrity, and the bays can be reconfigured for different module sizes or tools.

In an alternate embodiment of the present invention there is a hybrid compounded robotic platform wherein the intelligent management unit implements a control algorithm that compensates for shifts in center-of-mass of the manipulator assembly as end-effectors or workpieces are exchanged, by adjusting base drive and joint torques in real time based on tool identity and mass properties.

This embodiment enhances operational stability by dynamically redistributing torque commands and base movement vectors to maintain balance. Optional implementations may utilize inertial measurement units, strain gauges, or load cells to refine compensation accuracy. The algorithm can learn from prior tasks to anticipate mass distribution changes and pre-adjust motion trajectories.

In an alternate embodiment of the present invention there is a hybrid compounded robotic platform wherein the support architecture includes a coupling interface by which an immobile support base can be detachably mounted on a mobile support base to form a combined base for task execution and then detached for redeployment.

This embodiment provides a versatile structural foundation for applications requiring both stability and transportability. The detachable coupling interface may employ mechanical latches, electromechanical clamps, or quick-release fasteners designed to transmit power and data between the joined bases. In some configurations, fluidic couplers may also be included to transfer coolant or pneumatic supply lines. The combination base arrangement allows the robot to perform precision operations in a stationary mode and then reconfigure for mobility, such as repositioning within a facility or moving between workstations.

In an alternate embodiment of the present invention there is a hybrid compounded robotic platform wherein the robotic manipulator arm is forearm-only, including a wrist assembly and an elbow assembly projected from the support architecture, and omitting a shoulder and an upper arm, or wherein the robotic manipulator arm is configured with the elbow assembly directly connected to the support architecture.

This embodiment achieves a compact, lightweight, and high-precision manipulator suited for close-range or confined-space tasks. The omission of shoulder and upper-arm joints simplifies control and reduces inertia, enabling faster actuation and more stable performance. Optional configurations may include replaceable elbow segments for variable reach, modular wrist adapters for different tool sizes, and sealed enclosures for sterile or outdoor environments.

In an alternate embodiment of the present invention there is a hybrid compounded robotic platform wherein at least one collaborative robot arm is configured as a primary end-effector detachably coupled to the wrist assembly or to a lifting column mounted on the support architecture, and wherein one or more secondary end-effectors are detachably mounted to the ccollaborative arm.

This embodiment enhances human-robot interaction capability and flexibility. The collaborative arm, serving as a primary end-effector, can provide additional axes of freedom for delicate handling or co-operative assembly tasks. The lifting column may be telescopic, providing variable height for ergonomic adaptation or overhead positioning. Secondary end-effectors attached to the collaborative arm may include grippers, sensors, or precision actuators, enabling multi-tool sequencing in a compact workspace.

In an alternate embodiment of the present invention there is a hybrid compounded robotic platform wherein the parking station is mounted on the manipulator arm or the wrist assembly to shorten tool travel paths, or is external and networked to allow multi-platform sharing of end-effectors or workpieces.

This configuration minimizes idle motion during tool exchanges and increases operational throughput. The on-arm station may be implemented as a compact rotary carousel, allowing rapid tool swaps within the manipulator's reach envelope. Alternatively, external stations can be networked among multiple robots via wireless or optical communication for shared access to tools and components. Optional designs may include automated inventory tracking and temperature-controlled storage for specialized tools.

In an alternate embodiment of the present invention there is a hybrid compounded robotic platform further comprising autonomous battery or power-module swapping at the parking station for any end-effector or workpiece that includes an on-board power source, the swapping triggered by the intelligent management unit based on state-of-charge thresholds.

This embodiment ensures uninterrupted operation by automating power management. The battery-swapping subsystem may utilize robotic actuators within the docking bay or an auxiliary manipulator arm dedicated to power exchange. The management unit continuously monitors charge status and schedules replacement cycles to coincide with calibration intervals. Optional configurations include hot-swappable power packs, inductive charging pads, or hybrid battery-super capacitor designs for rapid re-energizing.

In an alternate embodiment of the present invention there is a hybrid compounded robotic platform further comprising a base-to-base multi-signal connector enabling two or more platforms to be physically coupled at their support bases to share power, data, and fluids and to perform cooperative operations as a unified system.

This embodiment establishes inter-platform synergy. The base-to-base connector integrates structural docking features and conductive or optical interfaces for high-bandwidth communication. Optional enhancements include a locking alignment latch, redundant power routing, and shared sensor fusion algorithms that synchronize platform movement and load balancing. When coupled, the unified system behaves as a single coordinated robotic entity capable of manipulating larger or more complex objects.

In a preferred embodiment of the present invention there is a system of robotic platforms comprising: a plurality of platforms each configured as a hybrid compounded robotic platform including a support architecture with mobile or immobile bases, a manipulator arm terminating in a wrist assembly, and a chain of primary, secondary, and tertiary end-effectors coupled via multi-signal connectors; and a central coordination server in networked communication with each platform's intelligent management unit, the server programmed to allocate tasks, synchronize end-effector or workpiece exchanges across multiple bases, and resolve inter-platform collision risks via shared position and trajectory data.

This embodiment establishes a distributed robotic network in which several hybrid platforms cooperate as a synchronized fleet. The central coordination server maintains wireless or optical links to each platform, issuing task assignments and coordinating shared workspace navigation. Optional configurations may include hierarchical task delegation where one platform functions as a supervisor node. The shared trajectory database ensures real-time motion planning and collision-free coordination during multi-unit operation.

In an alternate embodiment of the present invention there is a system of robotic platforms wherein the central coordination server maintains a dynamic map of each platform's support architecture state, parking-station inventory, tool identity, and battery status, and autonomously directs a first platform to deliver a specified secondary end-effector or workpiece to a second platform's parking station for exchange.

This embodiment enables active resource sharing. The dynamic map is continuously updated through sensor feedback and communication protocols such as MQTT or ROS-based frameworks. By monitoring energy status and tool inventory, the server ensures optimal task distribution and prevents redundant tool assignments. Optional implementations allow predictive scheduling where upcoming tasks trigger pre-emptive tool transfers between units.

In an alternate embodiment of the present invention there is a system of robotic platforms wherein end-effector or workpiece exchange between platforms is dynamic or static, including direct manipulator-to-manipulator transfer or parking-station-mediated transfer, with verification of alignment and coupler engagement prior to release.

This embodiment provides operational flexibility. Dynamic exchanges occur when two robotic arms align and pass a tool or object directly, whereas static exchanges involve docking stations that act as intermediaries. Optional verification systems may include proximity sensors, torque feedback, or optical fiducials to confirm accurate engagement before transfer. The ability to choose exchange mode according to the workspace environment enhances both speed and reliability of cooperative operations.

In an alternate embodiment of the present invention there is a system of robotic platforms wherein the central coordination server can command temporary physical coupling of two or more platforms via the base-to-base multi-signal connector to enable load sharing, synchronized motion, or extended-reach operations.

This configuration allows mechanical and data integration between platforms for large-scale or heavy-duty tasks. When coupled, their control systems merge under the server's coordination algorithm, sharing torque feedback and positional data for real-time synchronization. Optional variations include smart latching systems that automatically align and electrically link the bases, creating a modular robotic structure capable of joint manipulation of extended workpieces or large patient supports.

In a preferred embodiment of the present invention there is a method of automated task execution using the hybrid compounded robotic platform comprising: receiving a task specification identifying a required tertiary end-effector or workpiece and a work location; commanding the intelligent management unit to decouple any end-effectors or workpieces not required for the task and dock them in the parking station for charging and calibration; retrieving and coupling the primary, secondary, and tertiary end-effectors or workpieces in sequence via their multi-signal couplers; compensating base and manipulator motions for shifting mass distribution resulting from end-effector or workpiece exchanges; performing the specified task at the work location; and upon task completion, returning end-effectors or workpieces to their respective docking bays for post-use calibration and power replenishment.

This method integrates mechanical sequencing, dynamic balancing, and autonomous scheduling. The task specification may be derived from a digital plan, a voice command, or external scheduling software. The management unit interprets the instruction, executes the sequence autonomously, and ensures continuous calibration of tool parameters. Optional enhancements include real-time learning modules that refine sequence efficiency based on prior performance data.

In an alternate embodiment of the present invention there is a method of automated task execution wherein receipt of the task specification comprises parsing a voice command, converting it to a structured task plan, and forwarding the plan wirelessly to the intelligent management unit.

This embodiment enables intuitive human-machine interaction. The system can accept voice commands through a microphone array or a wireless interface connected to the platform or server. Natural-language processing modules translate spoken instructions into executable task plans defining target coordinates, tool selections, and sequencing order. Optional configurations include multi-language support, gesture-linked confirmation, or augmented-reality overlays to visualize the planned task before execution. The voice interface can also serve as a safety feature, allowing operators to issue emergency stop or override commands.

In an alternate embodiment of the present invention there is a method of automated task execution further comprising inter-platform exchange in which the central coordination server directs a first platform to deliver a designated secondary end-effector or workpiece to a second platform, including alignment verification, coupler engagement, and inventory update in the dynamic map.

This embodiment provides distributed resource sharing among multiple robotic units. The coordination server determines which platform currently possesses the needed end-effector and autonomously plans a transfer route. Alignment verification may involve optical fiducials, RFID tracking, or laser triangulation. Upon verified coupler engagement, the inventory database updates automatically to reflect the new tool location. Optional safety protocols prevent duplicate claims of the same resource or unverified coupling events.

In an alternate embodiment of the present invention there is a method of automated task execution wherein autonomous power-module swapping is performed for a tool or workpiece that requires replenishment, the swapping coordinated with tool calibration routines so that the tool is returned to service in a ready-to-use state.

This embodiment merges energy management with maintenance scheduling. When a tool's charge or pressure level falls below a programmed threshold, the intelligent management unit initiates an automatic swap or recharge cycle. The process occurs concurrently with calibration and diagnostics, reducing downtime. Optional configurations include hot-swap trays, dual-battery packs, or quick-change gas canisters for pneumatic devices. By combining calibration and energy renewal, operational continuity and precision are both ensured.

In an alternate embodiment of the present invention there is a method of automated task execution wherein the support architecture is configured from an immobile base detachably mounted on a mobile base for the task, and is detached after completion for redeployment to a new location.

This embodiment allows modular deployment in facilities where tasks occur in distributed zones. The mobile base transports the immobile upper module between workstations, then detaches to allow independent fixed-base operation. Optional mechanical arrangements include electromagnetic locks, mechanical clamps, or guided rails. Communication between bases is maintained through the multi-signal coupling system to transfer control and power seamlessly during attachment or detachment. The design enhances portability while preserving high-precision operation once stationed.

In an alternate embodiment of the present invention there is a hybrid compounded robotic platform wherein the support base is omni-form to accommodate and stow different shapes of end-effectors or workpieces, including ring-type devices and patient-support articles, thereby enabling tool or workpiece parking and transport as part of task execution.

This embodiment introduces a universal support geometry adaptable to diverse module shapes. The omni-form base may feature interchangeable inserts, retractable clamps, or adjustable docking contours to secure ring-style imaging heads, flat trays, or elongated components. The same structural frame can serve as both transporter and storage cradle. Optional sensors embedded in the docking interfaces monitor load distribution and confirm retention during motion. The omni-form configuration increases system adaptability across industrial, medical, and logistics domains.

The present invention provides a unified robotic ecosystem capable of performing diverse and sequential operations through modular reconfiguration. Unlike conventional robotic systems that are fixed-function and tool-specific, the hybrid compounded platform adapts dynamically to varied tasks through its compound end-effector architecture and intelligent management algorithms.

An advantage of the present invention is that it allows rapid and automated tool exchanges through its multi-signal coupler system, which supports mechanical, electrical, and fluidic connectivity in a single unified interface. This significantly reduces manual intervention, downtime, and reprogramming requirements between task transitions.

Another advantage is the intelligent management unit's ability to compensate in real time for changes in balance, torque, and mass distribution as tools are exchanged. This feature maintains stability during motion, enhancing both safety and precision in environments where variable tool configurations could otherwise disrupt control performance.

The parking station and associated charging and calibration systems provide an additional advantage by enabling continuous operation and readiness. End-effectors or workpieces are automatically charged, tested, and calibrated when docked, creating a self-maintaining ecosystem that minimizes human maintenance and operational interruptions.

The modular base configuration—comprising mobile, immobile, and detachable combinations—offers unique flexibility. It allows the platform to operate as a stationary precision robot or a mobile manipulator depending on the operational requirement. This adaptability makes it suitable for both laboratory-grade precision applications and industrial field deployment.

The cooperative multi-platform system and networked coordination server further expand the utility of the invention. Multiple platforms can collaborate through synchronized data sharing and task allocation, allowing distributed robotics networks to perform large-scale or complex multi-stage operations with centralized oversight.

The inclusion of voice-command parsing and structured task planning capabilities enhances human-machine integration, allowing intuitive control and adaptive scheduling. This advantage extends the system's usability beyond industrial automation to collaborative work environments where safety and natural interaction are paramount.

The autonomous power-management functions, including battery or power-module swapping, ensure sustained uptime. This makes the platform particularly advantageous in continuous-operation scenarios such as hospital logistics, automated manufacturing, or hazardous environments where manual intervention is impractical or unsafe.

The system's capacity for inter-platform coupling and cooperative manipulation through the base-to-base connector provides an additional advantage by enabling load sharing and extended-reach operations. Two or more platforms can synchronize to lift or transport large, delicate, or heavy components, thereby increasing payload capability without requiring larger individual robots.

The omni-form support base further enhances versatility by allowing docking, storage, and transport of end-effectors or workpieces of varying shapes and sizes. This broadens the system's adaptability to include ring-shaped devices, patient supports, or irregular workpieces, supporting use in medical, research, and manufacturing contexts.

Another advantage is the invention's built-in scalability across industries. The same core system architecture may be configured for surgical automation, manufacturing assembly, logistics handling, or environmental inspection simply by interchanging modular components and updating software parameters.

The hybrid compounded robotic platform's design also provides sustainability benefits. By reusing modular components and eliminating the need for multiple dedicated systems, material waste and total lifecycle cost are reduced. Maintenance tasks are simplified, and software-driven updates extend the useful life of the hardware.

In combination, the features of the present invention provide an integrated, intelligent, and reconfigurable robotic platform that unifies the capabilities of multiple specialized machines into a single adaptable system. It achieves superior operational continuity, task diversity, and human-interactive safety while maintaining precision and reliability across multiple application domains.

Overall, the invention establishes a foundational framework for next-generation compound robotics, combining mechanical modularity, autonomous control intelligence, and cooperative networking into a scalable, self-managing platform architecture suitable for industrial, medical, and research applications worldwide.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1. A hybrid compounded robotic platform comprising:
a support architecture including a mobile support base, an immobile support base, or a detachable combination thereof;
at least one robotic manipulator arm mounted to said support architecture and terminating in a wrist assembly;
a primary end-effector or a workpiece detachably coupled to said wrist assembly via a first multi-signal coupler that provides mechanical, electrical, and fluidic connections;
a secondary end-effector detachably coupled to said primary end-effector via a second multi-signal coupler;
a tertiary end-effector detachably coupled to said secondary end-effector via a third multi-signal coupler;
wherein said primary end-effector includes actuators configured to drive movement of said secondary end-effector, and said secondary end-effector includes actuators configured to drive movement of said tertiary end-effector;
a parking station integrated into or on said support architecture or implemented as an external station, said parking station including shaped docking seats and alignment fixtures matching each of said primary end-effector, said secondary end-effector, and said tertiary end-effector or said workpiece, and further including electrical contacts for automatic charging and calibration when docked; and
an intelligent management unit in communication with said robotic manipulator arm, said multi-signal coupler, and said parking station, said intelligent management unit programmed to automatically sequence coupling and decoupling of said primary end-effector, said secondary end-effector, and said tertiary end-effector or said workpiece based on a received task instruction, a monitor power, a balance sensor, and a collision-avoidance sensor and adjust said robotic manipulator arm and said mobile support base motions in real time as said primary end-effector, said secondary end-effector, and said tertiary end-effector or said workpiece are exchanged, and command said parking station to charge, calibrate, self-test, or swap a power module for any docked end-effector or said workpiece.

2. The platform of claim 1, wherein said first multi-signal coupler, said second multi-signal coupler, or said third multi-signal coupler comprises an interlocking mechanical latch, a plurality of electrical power and data contacts, and one or more fluidic conduits, each arranged to automatically mate when halves of said multi-signal coupler engage, with tapered guides and alignment pins to ensure a proper orientation.

3. The platform of claim 1, wherein each of said primary end-effector, said secondary end-effector, and said tertiary end-effector in a chain includes at least one jointed degree of freedom that is rotational or prismatic, and a dedicated drive motor, such that said dedicated drive motor of said primary end-effector drives a positioning of said secondary end-effector in at least one axis independent of said robotic manipulator arm.

4. The platform of claim 1, wherein said primary end-effector, said secondary end-effector, or said tertiary end-effector comprises a medical device, a manufacturing tool, a logistics tool, a construction tool, an inspection or imaging device, or a collaborative robot arm.

5. The platform of claim 1, wherein said parking station comprises three individualized docking bays, each shaped to a geometry of one of said primary end-effector, said secondary end-effector, and said tertiary end-effector or said workpiece, and further comprising passive alignment pins and spring-loaded electrical contacts for auto-alignment and reliable electrical engagement.

6. The platform of claim 1, wherein said intelligent management unit implements a control algorithm that compensates for shifts in center-of-mass of said robotic manipulator arm as said primary end-effector, said secondary end-effector, and said tertiary end-effector or said workpiece are exchanged, by adjusting base drive and joint torques in real time based on tool identity and mass properties.

7. The platform of claim 1, wherein said support architecture includes a coupling interface by which said immobile support base can be detachably mounted on said mobile support base to form a combined base for task execution and then detached for redeployment.

8. The platform of claim 1, wherein said robotic manipulator arm is forearm-only, including said wrist assembly and an elbow assembly projected from said support architecture, and omitting a shoulder and an upper arm, or wherein said robotic manipulator arm is configured with said elbow assembly directly connected to said support architecture.

9. The platform of claim 1, wherein at least one collaborative robot arm is configured as said primary end-effector detachably coupled to said wrist assembly or to a lifting column mounted on said support architecture, and wherein one or more secondary end-effectors are detachably mounted to said collaborative robot arm.

10. The platform of claim 1, wherein said parking station is mounted on said robotic manipulator arm or said wrist assembly to shorten tool travel paths, or is external and networked to allow multi-platform sharing of said primary end-effector, said secondary end-effector, and said tertiary end-effector or said workpiece.

11. The platform of claim 1, further comprising an autonomous battery or power module swapping at said parking station for any said primary end-effector, said secondary end-effector, or said tertiary end-effector or said workpiece that includes an on-board power source, said autonomous battery or power-module swapping triggered by said intelligent management unit based on state-of-charge thresholds.

12. The platform of claim 1, further comprising a base-to-base multi-signal connector enabling two or more platforms to be physically coupled at said mobile support base or said immobile support base to share power, data, and fluids and to perform cooperative operations as a unified system.

13. A system of robotic platforms comprising:
a plurality of platforms according to claim 1; and
a central coordination server in networked communication with an intelligent management unit of each of said plurality of platforms including at least a first platform and a second platform, said central coordination server programmed to allocate tasks, synchronize an end-effector or workpiece exchange across multiple bases, and resolve inter-platform collision risks via shared position and trajectory data.

14. The system of claim 13, wherein said central coordination server maintains a dynamic map of a support architecture state of each of said plurality of platforms, a parking-station inventory, a tool identity, and a battery status, and autonomously directs said first platform to deliver a specified secondary end-effector or a workpiece to a parking station of a second platform for exchange.

15. The system of claim 13, wherein said end-effector or workpiece exchange between said plurality of platforms is dynamic or static, including direct manipulator-to-manipulator transfer or parking-station mediated transfer, with verification of alignment and coupler engagement prior to release.

16. The system of claim 13, wherein said central coordination server can command temporary physical coupling of two or more of said plurality of platforms via a base-to-base multi-signal connector to enable load sharing, synchronized motion, or extended reach operations.

17. A method of automated task execution using the platform of claim 1, comprising:
receiving a task specification identifying a tertiary end-effector or a workpiece and a work location;
commanding an intelligent management unit to decouple said tertiary end-effector or said workpiece not required for a task and dock said tertiary end-effector or said workpiece in a parking station for charging and calibration;
retrieving and coupling a primary end-effector, a secondary end-effector, and said tertiary end-effector or said workpiece in sequence via a multi-signal coupler of each of said primary end-effector, said secondary end-effector, and said tertiary end-effector;
compensating base and manipulator motions for shifting mass distribution resulting from any of said primary end-effector, said secondary end-effector, or said tertiary end-effector or workpiece exchange;
performing said task at said work location; and
upon a task completion, returning said primary end-effector, said secondary end-effector, and said tertiary end-effector or said workpiece to respective docking bays for post-use calibration and power replenishment.

18. The method of claim 17, wherein a receipt of said task specification comprises parsing a voice command, converting it to a structured task plan, and forwarding said structured task plan wirelessly to said intelligent management unit.

19. The method of claim 17, further comprising an inter-platform exchange in which a central coordination server directs a first platform to deliver said secondary end-effector or said workpiece to a second platform, including an alignment verification, a coupler engagement, and an inventory update in a dynamic map.

20. The method of claim 17, wherein an autonomous power-module swapping is performed for a tool or said workpiece that requires replenishment, said autonomous power-module swapping is coordinated with tool calibration routines so that said tool is returned to service in a ready-to-use state.

21. The method of claim 17, wherein a support architecture is configured from an immobile base detachably mounted on a mobile base for said task, and is detached after completion for redeployment to a new location.

22. The platform of claim 1, wherein said support architecture is omni-form to accommodate and stow different shapes of said primary end-effector, said secondary end-effector, and said tertiary end-effector or said workpiece, including ring-type devices and patient-support articles, thereby enabling tool or workpiece parking and transport as part of task execution.
